(19) United States
(12) Patent Application Publication
(10) Pub. No.: US 2023/0165690 A1
(43) Pub. Date: Jun. 1, 2023

(54) SPINAL IMPLANT DEVICE

(71) Applicant: Spinal Elements, Inc., Carlsbad, CA (US)

(72) Inventors: Jason Blain, Encinitas, CA (US); Samuel Tolpen, San Diego, CA (US); David Ortiz, Oceanside, CA (US); Christiana Gabrielle Salvosa, San Diego, CA (US)

(21) Appl. No.: 18/056,006

(22) Filed: Nov. 16, 2022

Related U.S. Application Data
(60) Provisional application No. 63/283,723, filed on Nov. 29, 2021.

(51) Int. Cl. A61F 2/44

(52) U.S. Cl. CPC A61F 2/447; A61F 2002/30593

(57) ABSTRACT

A spinal implant device is provided comprising a body structure and a movable door coupled or configured to couple to the body structure. The spinal implant device can include a distal end, a proximal end, two opposing side walls extending between the distal end and the proximal end, an upper wall, a lower wall, and a central cavity. The movable door is configured to open to allow the central cavity to be packed with material and the movable door is configured to close. A method is provided comprising providing a spinal implant device, packing the cavity with the material, closing the movable door, and inserting the spinal implant device between adjacent vertebrae. A method of manufacturing a spinal implant device is provided comprising 3D printing a spinal implant device, wherein the spinal implant device is 3D printed as one piece.

### CROSS-REFERENCE TO RELATED APPLICATIONS

This application claims priority benefit to U.S. Provisional Pat. Application No. 63/283723, filed Nov. 29, 2021, the entirety of which is hereby incorporated by reference herein.

### BACKGROUND

### Field

Some embodiments described herein relate generally to systems and methods for stabilizing bone, for example, stabilizing vertebrae by fusing adjacent vertebrae. Some embodiments relate to spinal implant devices, for example spinal implant devices with a structure to enhance rapid bone growth.

### Description of the Related Art

Advancing age, as well as injury, can lead to degenerative changes in the bones, discs, joints, and ligaments of the spine. These degenerative changes can produce pain and spinal instability. Under certain circumstances, spinal fusion can alleviate these degenerative changes. Spinal fusion is a surgical technique in which two or more vertebrae of the spinal column are fused together to reduce or limit the motion between the vertebrae. Spinal fusion is used to treat various conditions including fracture, scoliosis, and spondylolisthesis. Spinal fusion with discectomy is used to treat herniation of the discs by removal of the affected disc and fusion of the adjacent vertebrae. There are several procedures available to patients with degenerative spine conditions.

One technique is called Anterior Cervical Discectomy and Fusion (“ACDF”). In the ACDF method, the spine is typically accessed through the throat area to access the disc. In this method, the neck muscles, trachea, and esophagus are moved to provide access. In this method, the disc is removed and a graft is inserted to fuse the vertebrae. Surgery from an anterior approach may be preferred over the posterior approach for cervical vertebrae. Another technique is called Anterior Lumbar Interbody Fusion (“ALIF”). In the ALIF method, the spine is approached from the front of the body of the patient to access the disc. In this method, the abdominal muscles are moved to provide access. In this method, the disc is removed and an implant is inserted to fuse the vertebrae. Surgery from an anterior approach may be preferred over the posterior approach for lumbar vertebrae since a larger implant can be inserted, which can provide better stability.

In some methods, a spinal implant device is inserted into the disc space to promote bone growth between adjacent vertebrae. The spinal implant device is positioned between adjacent vertebrae in the disc space. In some methods, the spinal implant device can be secured to one or more vertebrae by bone screws or other similar fasteners inserted through holes in the spinal implant. The size of the spinal implant device is typically selected such that the spinal implant device forces the vertebrae apart to cause tensing of the vertebral annulus and other soft tissue structures surrounding the joint space. Tensing the soft tissues surrounding the joint space can result in the vertebrae exerting compressive forces on the spinal implant device to maintain the spinal implant device in place.

The current standard of care to address the degenerative changes of the spine is to fuse vertebrae. The relative motion between the two adjacent vertebrae is limited or reduced in some methods of use.

### SUMMARY

In some embodiments, a spinal implant device is provided. The spinal implant device can include a body structure. The spinal implant device can include a movable door coupled or configured to couple to the body structure. In some embodiments, the spinal implant device comprises a distal end, a proximal end, two opposing side walls extending between the distal end and the proximal end, an upper wall, a lower wall, and a central cavity. In some embodiments, the movable door is configured to open to allow the central cavity to be packed with material. In some embodiments, the movable door is configured to close.

In some embodiments, the two opposing side walls comprise porous or network surfaces. In some embodiments, the upper wall and the lower wall comprise porous or network surfaces. In some embodiments, the proximal end comprises a threaded opening. In some embodiments, the upper wall and the lower wall comprise ridges. In some embodiments, the body structure comprises a first pin and a second pin. In some embodiments, the movable door is coupled to the body structure with a hinge. In some embodiments, the movable door is coupled to the body structure with a pin extending vertically between the upper wall and the lower wall. In some embodiments, the movable door is configured to pivot toward and away from the body structure. In some embodiments, the movable door is coupled to the body structure with a pin extending horizontally between the movable door and the body structure. In some embodiments, the movable door is configured to pivot from a generally transverse orientation to a generally vertical orientation. In some embodiments, the body structure comprises a captive pin formed by 3D printing. In some embodiments, the movable door comprises a catch configured to engage a pin when the moveable door is closed. In some embodiments, the movable door comprises a hinge aligned along a side wall of the two opposing side walls. In some embodiments, the body structure comprises a first protrusion and a second protrusion for securing the movable door. In some embodiments, the movable door comprises a first tab and a second tab configured to engage the first protrusion and the second protrusion when the moveable door is closed. In some embodiments, the movable door comprises a first catch and a second catch configured to form a snap fit with the first protrusion and the second protrusion when the moveable door is closed.

In some embodiments, a method is provided. The method can include providing a spinal implant device. The method can include packing the cavity with the material. The method can include closing the movable door. The method can include inserting the spinal implant device between adjacent vertebrae. In some embodiments, the material is a graft material.

In some embodiments, a method of manufacturing a spinal implant device is provided. The method can include 3D printing a spinal implant device. The spinal implant device can include a body structure. The spinal implant device can include a movable door coupled to the body structure. In some embodiments, the spinal implant device comprises a distal end, a proximal end, two opposing side walls extending between the distal end and the proximal end, an upper wall, a lower wall, and a central cavity. In some embodiments, the movable door is configured to open to allow the central cavity to be packed with material. In some embodiments, the movable door is configured to close. In some embodiments, the spinal implant device is 3D printed as one piece.

Accordingly, a need exists for a spinal implant device to quickly and/or easily stabilize and/or fixate vertebrae for facilitating fusion.

### BRIEF DESCRIPTION OF THE DRAWINGS

The structure and method of use will be better understood with the following detailed description of embodiments, along with the accompanying illustrations, in which:

FIG. 1 is a perspective view of an embodiment of a spinal implant device with a movable door shown in a closed position.

FIG. 2 is a distal view of the spinal implant device of FIG. 1.

FIG. 3 is a proximal view of the spinal implant device of FIG. 1.

FIG. 4 is a side view of the spinal implant device of FIG. 1.

FIG. 5 is another side view of the spinal implant device of FIG. 1.

FIG. 6 is a top view of the spinal implant device of FIG. 1.

FIG. 7 is a bottom view of the spinal implant device of FIG. 1.

FIG. 8 is a top perspective view of the spinal implant device of FIG. 1 with the movable door shown in an opened position.

FIG. 9 is an exploded perspective view of the spinal implant device of FIG. 1.

FIG. 10 is a perspective view of an embodiment of a spinal implant device with a movable door shown in a closed position.

FIG. 11 is a distal view of the spinal implant device of FIG. 10.

FIG. 12 is a proximal view of the spinal implant device of FIG. 10.

FIG. 13 is a side view of the spinal implant device of FIG. 10.

FIG. 14 is another side view of the spinal implant device of FIG. 10.

FIG. 15 is a top view of the spinal implant device of FIG. 10.

FIG. 16 is a bottom view of the spinal implant device of FIG. 10.

FIG. 17 is a top perspective view of the spinal implant device of FIG. 10 with the movable door shown in an opened position.

FIG. 18 is another top perspective view of the spinal implant device of FIG. 10 with the movable door shown in an opened position.

FIG. 19 is an exploded perspective view of the spinal implant device of FIG. 10.

FIG. 20 is a perspective view of an embodiment of a spinal implant device with a movable door shown in a closed position.

FIG. 21 is a distal view of the spinal implant device of FIG. 20.

FIG. 22 is a proximal view of the spinal implant device of FIG. 20.

FIG. 23 is a side view of the spinal implant device of FIG. 20.

FIG. 24 is another side view of the spinal implant device of FIG. 20.

FIG. 25 is a top view of the spinal implant device of FIG. 20.

FIG. 26 is a bottom view of the spinal implant device of FIG. 20.

FIG. 27 is a top perspective view of the spinal implant device of FIG. 20 with the movable door shown in an opened position.

FIG. 28 is another top perspective view of the spinal implant device of FIG. 20 with the movable door shown in an opened position.

### DETAILED DESCRIPTION

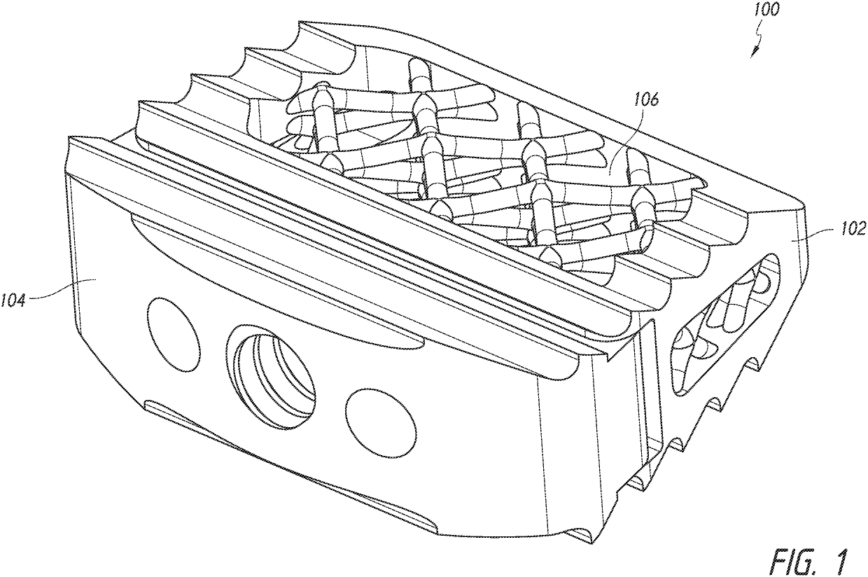

FIG. 1 illustrates a perspective view of a spinal implant device 100. The spinal implant device 100 can include a body structure 102 which can be placed between adjacent vertebral bodies. The body structure 102 can form a portion of the spinal implant device 100. The body structure 102 can form a portion of the length of the spinal implant device 100. The spinal implant device 100 can include a movable door 104. The movable door 104 can couple to the body structure 102, as described herein. The movable door 104 can allow the spinal implant device 100 to be packed with material, as described herein. The body structure 102 and the movable door 104 can define a cavity 106. The spinal implant device 100 can be designed for fusion as described herein. The spinal implant device 100 can include one or more rounded corners or edges. The spinal implant device 100 can include one or more sharp corners or edges. The spinal implant device 100 can include any feature described herein.

The spinal implant device 100 can be configured for insertion between any two vertebrae. The spinal implant device 100 can be configured for insertion between two cervical vertebrae. The spinal implant device 100 can be configured for insertion between two lumbar vertebrae. The spinal implant device 100 can be configured for insertion between two thoracic vertebrae. The spinal implant device 100 can be configured for any approach. The spinal implant device 100 can be configured for an anterior approach. The spinal implant device 100 can be configured for a posterior approach.

FIG. 2 is a distal view of the spinal implant device 100. The spinal implant device 100 can include a distal end 110. In some methods of use, the distal end 110 can be inserted into the space between adjacent vertebrae before another portion of the spinal implant device 100. In some embodiments, the distal end 110 forms a blunt or atraumatic shape. The distal end 110 can form a leading edge 112. The distal end 110 can include one or more sharp corners or edges. The distal end 110 can taper downward along an upper surface. The distal end 110 can taper upward along a lower surface. The distal end 110 can be rounded toward the sides of the spinal implant device 100. The distal end 110 can have a first radius of curvature extending from a first side surface. The distal end 110 can have a second radius of curvature extending from a second side surface. The first radius of curvature and the second radius of curvature can be the same. The first radius of curvature and the second radius of curvature can be different. The body structure 102 can include the distal end 110.

In some embodiments, the spinal implant device 100 can include a slight inclination toward one side of the spinal implant device 100. The spinal implant device 100 can have a lordosis angle. The spinal implant device 100 can have a lordosis angle to correspond to the natural orientation of the vertebral endplates. The lordosis angle can be zero. The lordosis angle can be an angle greater than zero. The lordosis angle can be an angle such as 0°, 1°, 2°, 3°, 4°, 5°, 6°, 7 °, 8°, 9°, 10°, 11°, 12°, 13°, 14°, 15°, 16°,17°, 18°, 19°, 20°, between 0° and 5°, between 0° and 6°, between 0° and 7°, between 3° and 9°, between 5° and 7°, between 6° and 12°, between 8° and 16°, between 10° and 14°, between 12° and 14°, between 16° and 20°, approximately 6°, approximately 12°, approximately 18°, or any range of two of the foregoing values. In some embodiments, the distal end 110 is tapered to one side by the lordosis angle as described herein.

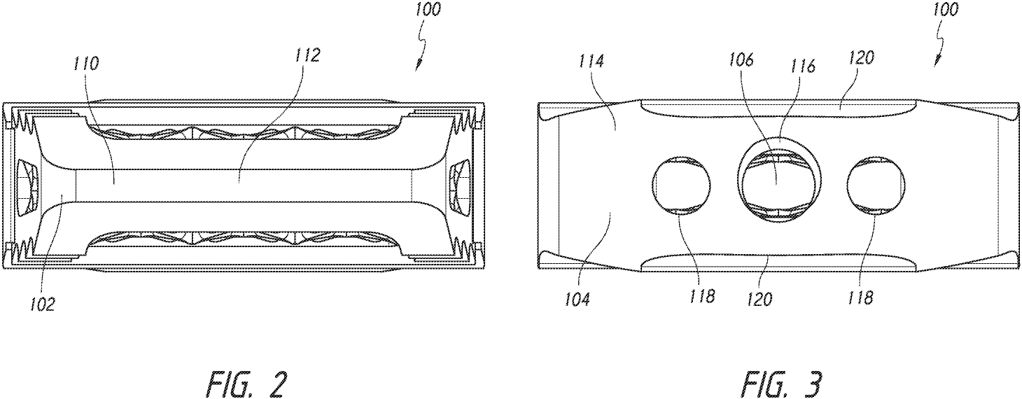

FIG. 3 is a proximal view of the spinal implant device 100. The spinal implant device 100 can include a proximal end 114. In some embodiments, the proximal end 114 is tapered to one side by the lordosis angle as described herein. The proximal end 114 can be a flat surface or generally flat surface. The proximal end 114 can be rounded toward the sides of the spinal implant device 100. The proximal end 114 can have a first radius of curvature extending from a first side surface. The proximal end 114 can have a second radius of curvature extending from a second side surface. The first radius of curvature and the second radius of curvature can be the same. The first radius of curvature and the second radius of curvature can be different. The movable door 104 can include the proximal end 114.

The proximal end 114 can engage with an insertion tool. The proximal end 114 can have any shape or surface that facilitates engagement with the insertion tool. In some embodiments, the spinal implant device 100 can include an opening 116. The opening 116 can be circular or rounded. In some embodiments, the opening 116 is located at the proximal end 114. The opening 116 can be through the movable door 104. The opening 116 can be threaded to engage a threaded end of the insertion tool. The opening 116 can be centrally located. The opening 116 can be located at a neutral center of the spinal implant device 100. The opening 116 can be located along the longitudinal axis of the spinal implant device 100. The opening 116 can extend into a cavity 106 of the spinal implant device 100.

The spinal implant device 100 can include one or more features 118 to facilitate placement of the spinal implant device 100. In some embodiments, the one or more features 118 are located at the proximal end 114. The one or more features 118 can be through the movable door 104. The feature 118 can be circular or rounded. The feature 118 can have at least one dimension that is smaller than the opening 116. The feature 118 can have a smaller height than the opening 116. The feature 118 can have a smaller width than the opening 116. The feature 118 can have a different shape than the opening 116. The one or more features 118 can be shaped to engage a complementary shaped end of the insertion tool. The one or more features 118 can be diametrically opposed relative to the opening 116. The one or more features 118 can be equally spaced relative to the opening 116. The one or more features 118 can extend into the cavity 106 of the spinal implant device 100. The opening 116 and the one or more features 118 can facilitate control of the spinal implant device 100. The opening 116 can prevent axial or translational movement between the spinal implant device 100 and the insertion tool. The one or more features 118 can prevent rotational movement between the spinal implant device 100 and the insertion tool. The proximal end 114 can include one or more undercuts 120. The top surface of the proximal end 114 can include the undercut 120. The lower surface of the proximal end 114 can include the undercut 120. The one or more undercuts 120 can facilitate engagement with the insertion tool. The one or more undercuts 120 can prevent rotational movement between the spinal implant device 100 and the insertion tool.

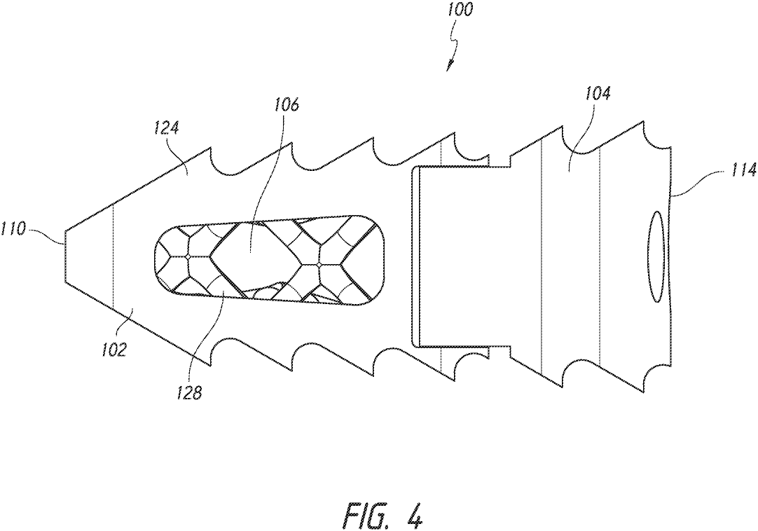

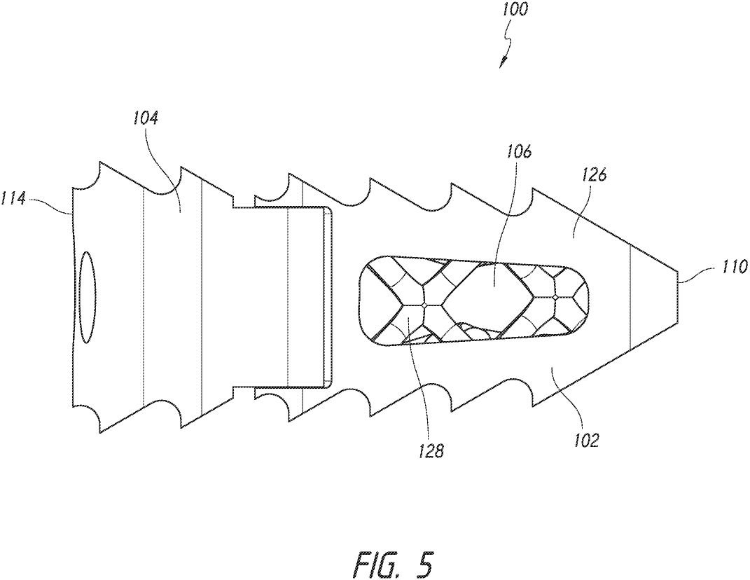

FIG. 4 is a side view of the spinal implant device 100. FIG. 5 is another side view of the spinal implant device 100. In some embodiments, the spinal implant device 100 can have a length measured between the distal end 110 and the proximal end 114. The spinal implant device 100 can include two side walls including a first side wall 124 and a second side wall 126. FIG. 4 illustrates the first side wall 124. FIG. 5 illustrates the second side wall 126. The first side wall 124 and the second side wall 126 can include the same dimensions. The first side wall 124 and the second side wall 126 can include the same features. The first side wall 124 and the second side wall 126 can be the same or similar. The first side wall 124 and the second side wall 126 can be identical. The first side wall 124 and the second side wall 126 can include different dimensions. The first side wall 124 and the second side wall 126 can include different heights due to the lordosis angle. The first side wall 124 and the second side wall 126 can include different features. The body structure 102 can include the first side wall 124. The body structure 102 can include the second side wall 126. The first side wall 124 can extend from the distal end 110 to the movable door 104. The second side wall 126 can extend from the distal end 110 to the movable door 104.

The first side wall 124 can extend along the length of the spinal implant device 100, or a portion thereof. The second side wall 126 can extend along the length of the spinal implant device 100, or a portion thereof. The first side wall 124 and the second side wall 126 can be opposing side walls. In some embodiments, the two side walls 124, 126 are parallel or generally parallel along at least a portion of the length of the spinal implant device 100. In some embodiments, the two side walls 124, 126 are aligned or generally aligned along at least a portion of the length of the spinal implant device 100.

The spinal implant device 100 can have a length or depth. The two side walls 124, 126 can define at least a portion of the length or depth of the spinal implant device 100. The movable door 104 can define at least a portion of the length or depth of the spinal implant device 100. The spinal implant device 100 can define a range of lengths. The spinal implant device 100 can have a maximum length of 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 20 mm, 21 mm, 22 mm, 23 mm, 24 mm, 25 mm, 26 mm, 27 mm, 28 mm, 29 mm, 20 mm, 30 mm, 31 mm, 32 mm, 33 mm, 34 mm, 35 mm, between 6 mm and 10 mm, between 10 mm and 15 mm, between 15 mm and 20 mm, between 23 mm and 27 mm, between 25 mm and 29 mm, between 27 mm and 31 mm, between 29 mm and 33 mm, or any range of two of the foregoing values. The length of the second side wall 126 can be less than the length of the first side wall 124. The length of the first side wall 124 can be less than the length of the second side wall 126. The first side wall 124 and the second side wall 126 can have the same or similar length.

In some embodiments, the two side walls 124, 126 can be spaced apart. The two side walls 124, 126 can define the width of the spinal implant device 100. The width can vary along the length. The two side walls 124, 126 can define a range of widths along at least a portion of the length of the spinal implant device 100. The maximum width as measured between the two side walls 124, 126 can be 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, 25 mm, 25.5 mm, 26 mm, 26.5 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, 35 mm, 35.5 mm, 36 mm, 36.5 mm, 37 mm, 37.5 mm, 38 mm, 38.5 mm, 39 mm, 39.5 mm, 40 mm, 40.5 mm, 41 mm, 41.5 mm, 42 mm, 42.5 mm, 43 mm, 43.5 mm, 44 mm, 44.5 mm, 45 mm, between 10 mm and 15 mm, between 14 mm and 16 mm, between 16 mm and 18 mm, between 17 mm and 20 mm, between 20 mm and 22 mm, between 28 mm and 32 mm, between 35 mm and 39 mm, between 39 mm and 43 mm, between 30 mm and 37 mm, between 30 mm and 41 mm, or any range of two of the foregoing values.

The two side walls 124, 126 can extend along the height of the spinal implant device 100. The two side walls 124, 126 can define the height of the spinal implant device 100. The height can vary based on the lordosis angle. The two side walls 124, 126 can define a range of heights. The first side wall 124 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The second side wall 126 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The height of the second side wall 126 can be less than the height of the first side wall 124. The lordosis angle can taper downward from the first side wall 124 toward the second side wall 126. The height of the first side wall 124 can be less than the height of the second side wall 126. The lordosis angle can taper downward from the second side wall 126 toward the first side wall 124.

In some embodiments, the first side wall 124 can include one or more porous or network surfaces 128. The one or more porous or network surfaces 128 can have any feature of the one or more porous or network surfaces described herein. The porous or network surfaces 128 can be a three-dimensional matrix. The porous or network surfaces 128 can include square, rectangular, or diamond openings or pores. The porous or network surfaces 128 can be generally non-planar. The porous or network surfaces 128 can include intersecting struts. The porous or network surfaces 128 can include a cross-linked structure. The porous or network surfaces 128 can be inter-linked. The porous or network surfaces 128 can be interwoven. The porous or network surfaces 128 can include struts that converge and diverge from one another. The porous or network surfaces 128 can include rows extending along one plane. The porous or network surfaces 128 can include alternating rows extending along another plane. The porous or network surfaces 128 can have openings. The porous or network surfaces 128 can have four sided openings. In some embodiments, at least two corners of an opening of the porous or network surfaces 128 lie on one plane. In some embodiments, at least two corners of the opening of the porous or network surfaces 128 lie on another plane. The porous or network surfaces 128 can include two layers. The two layers can merge at two corners of the opening. The two layers can form a depressed or lower set of corners. The two layers can diverge at two other corners of the opening. The two layers can form a bulged or upper set of corners.

The porous or network surfaces 128 can comprise a lattice unit cell. The porous or network surfaces 128 can include a narrow intersecting shape when viewed from the proximal end 114. The porous or network surfaces 128 can include a narrow X shape when viewed from the proximal end 114. The porous or network surfaces 128 can include separation of the arms smaller than the length of the arms. In some embodiments, the separation of the arms viewed from the proximal end 114 is 0.010 inch, 0.015 inch, 0.020 inch, 0.025 inch, 0.030 inch, 0.035 inch, 0.040 inch, 0.045 inch, 0.050 inch, or any range of two of the foregoing values. In some embodiments, the length of the arms is 0.70 inch, 0.075 inch, 0.080 inch, 0.085 inch, 0.090 inch, 0.095 inch, 0.100 inch, 0.105 inch, 0.110 inch, 0.115 inch, 0.120 inch, 0.125 inch, 0.130 inch, 0.135 inch, 0.140 inch, 0.145 inch, 0.150 inch, or any range of two of the foregoing values.

The porous or network surfaces 128 can include a wider intersecting shape when viewed from the first side 124 or the second side 126. The porous or network surfaces 128 can include a wide X shape when viewed from the first side 124 or the second side 126. The porous or network surfaces 128 can include separation of the arms about equal to the length of the arms. In some embodiments, the separation of the arms when viewed from the first side 124 or the second side 126 is 0.70 inch, 0.075 inch, 0.080 inch, 0.085 inch, 0.090 inch, 0.095 inch, 0.100 inch, 0.105 inch, 0.110 inch, 0.115 inch, 0.120 inch, 0.125 inch, 0.130 inch, 0.135 inch, 0.140 inch, 0.145 inch, 0.150 inch, or any range of two of the foregoing values. The porous or network surfaces 128 can include a diamond or rectangular orifice between adjacent intersecting shapes when viewed from the first side 124 or the second side 126. In some embodiments, the diagonal dimension of the orifice is 0.035 inch, 0.040 inch, 0.045 inch, 0.050 inch, 0.055 inch, 0.060 inch, 0.70 inch, 0.075 inch, 0.080 inch, 0.085 inch, 0.090 inch, 0.095 inch, 0.100 inch, or any range of two of the foregoing values. The porous or network surfaces 128 can include struts. The porous or network surfaces 128 can include intersecting struts. The struts can include a cross-sectional dimension of 0.005 inch, 0.010 inch, 0.015 inch, 0.020 inch, 0.025 inch, 0.030 inch, or any range of two of the foregoing values. The struts can be thin and elongate. The struts can intersect at a junction. The junction can be bulbous or rounded. The junction can have a radius of curvature.

In some embodiments, the openings of porous or network surfaces 128 can cover a percentage of the surface of the porous or network surfaces 128 such as at least 5%, 10%, 15%, 20%, 25%, 30%, 35%, 40%, 45%, 50%, 55%, 60%, 65%, 70%, 75% or any range of two of the foregoing values. In some embodiments, the struts or structure of porous or network surfaces 128 can cover a percentage of the surface of the porous or network surfaces 128 such as at least 5%, 10%, 15%, 20%, 25%, 30%, 35%, 40%, 45%, 50%, 55%, 60%, 65%, 70%, 75% or any range of two of the foregoing values.

The first side wall 124 can include one or more distinct sections of porous or network surfaces 128. The first side wall 124 can include one distinct section of porous or network surfaces 128. The porous or network surfaces 128 can extend along the length of the first side wall 124. In some embodiments, the porous or network surfaces 128 can cover a percentage of the first side wall 124 such as at least 5%, 10%, 15%, 20%, 25%, 30%, 35%, 40%, 45%, 50%, 55%, 60%, 65%, 70%, 75% or any range of two of the foregoing values. The porous or network surfaces 128 can be generally linear. The first side wall 124 can be generally linear along the length. In some embodiments, the first side wall 124 can be concave along the length. In some embodiments, the first side wall 124 can be convex along the length. The sections of the porous or network surfaces 128 of the first side wall 124 can mirror the curvature of first side wall 124.

The porous or network surfaces 128 can facilitate the load bearing capacity of the first side wall 124. The one or more porous or network surfaces 128 can have the same width or smaller width than another portion of the first side wall 124. The first side wall 124 can form a frame around the porous or network surfaces 128. The frame of the first side wall 124 around the porous or network surfaces 128 can facilitate the load bearing capacity of the first side wall 124. The one or more porous or network surfaces 128 can have the same width or smaller width than the corresponding edges. In some embodiments, the first side wall 124 does not include one or more porous or network surfaces. In some embodiments, the first side wall 124 is solid. In some embodiments, the first side wall 124 is open. In some embodiments, the first side wall 124 is not porous. In some embodiments, the first side wall 124 can have any features described herein.

The second side wall 126 can include one or more distinct sections of porous or network surfaces 128. The second side wall 126 can include one distinct section of porous or network surfaces 128. The porous or network surfaces 128 can extend along the length of the second side wall 126. In some embodiments, the porous or network surfaces 128 can cover a percentage of the second side wall 126 such as at least 5%, 10%, 15%, 20%, 25%, 30%, 35%, 40%, 45%, 50%, 55%, 60%, 65%, 70%, 75% or any range of two of the foregoing values. The porous or network surfaces 128 can be generally linear. The second side wall 126 can be generally linear along the length. In some embodiments, the second side wall 126 can be concave along the length. In some embodiments, the second side wall 126 can be convex along the length. The sections of the porous or network surfaces 128 of the second side wall 126 can mirror the curvature of second side wall 126.

The porous or network surfaces 128 can facilitate the load bearing capacity of the second side wall 126. The one or more porous or network surfaces 128 can have the same width or smaller width than another portion of the second side wall 126. The second side wall 126 can form a frame around the porous or network surfaces 128. The frame of the second side wall 126 around the porous or network surfaces 128 can facilitate the load bearing capacity of the second side wall 126. The one or more porous or network surfaces 128 can have the same width or smaller width than the corresponding edges. In some embodiments, the second side wall 126 does not include one or more porous or network surfaces. In some embodiments, the second side wall 126 is solid. In some embodiments, the second side wall 126 is open. In some embodiments, the second side wall 126 is not porous. In some embodiments, the second side wall 126 can have any features described herein.

The porous or network surfaces 128 can be the same matrix on the first side wall 124 and the second side wall 126. The porous or network surfaces 128 of the first side wall 124 and the second side wall 126 can have the same length or generally the same length. The porous or network surfaces 128 of the first side wall 124 and the second side wall 126 can have the same width or generally the same width. The porous or network surfaces 128 of the first side wall 124 and the second side wall 126 can have the same height or generally the same height. The porous or network surfaces 128 of the first side wall 124 and the second side wall 126 can create a passageway in a horizontal direction. The one or more porous or network surfaces 128 can allow ingrowth as described herein. The porous or network surfaces 128 of the first side wall 124 and the second side wall 126 can promote fusion into the cavity 106.

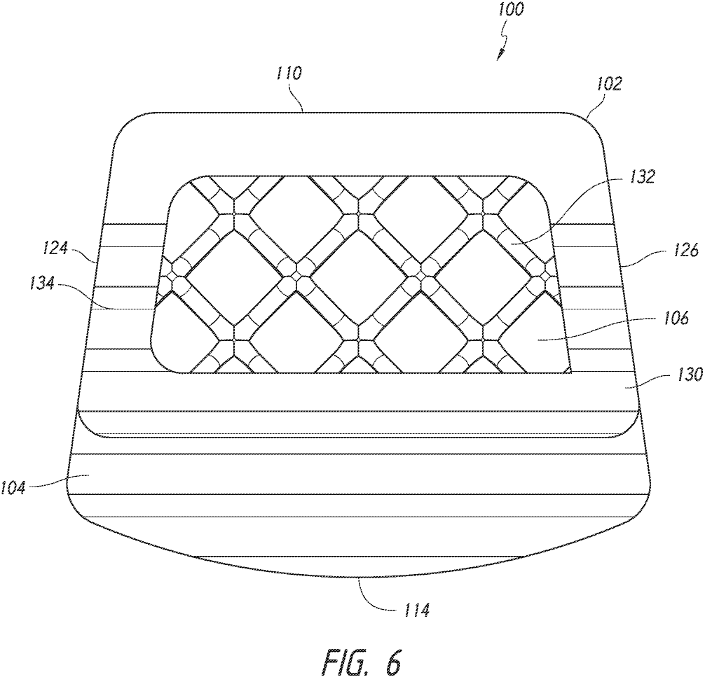

FIG. 6 is a top view of the spinal implant device 100. The two opposing side walls 124, 126 can extend between the distal end 110 and the proximal end 114. In some embodiments, the two opposing side walls 124, 126 are separated by a similar width along a majority of the length of the two opposing side walls 124, 126. In some embodiments, the two opposing side walls 124, 126 are separated by a varying width along a majority of the length of the two opposing side walls 124, 126. In some embodiments, the two opposing side walls 124, 126 generally taper along a majority of the length of the side walls 124, 126. In some embodiments, the two opposing side walls 124, 126 are generally at an angle to each other along a majority of the length of the side walls 124, 126. In some embodiments, the two opposing side walls 124, 126 taper to the distal end 110. In some embodiments, the two opposing side walls 124, 126 have a smaller width near the distal end 110 than the proximal end 114. In some embodiments, the first side wall 124 is straight or generally straight along a majority of the length of the first side wall 124. In some embodiments, the second side wall 126 is straight or generally straight along a majority of the length of the second side wall 126. In some embodiments, the two opposing side walls 124, 126 are slightly curved along a majority of the length of the two opposing side walls 124, 126. The two opposing side walls 124, 126 can bow outward. The two opposing side walls 124, 126 can bow inward. The body structure 102 can include the two opposing side walls 124, 126. The two opposing side walls 124, 126 can extend toward the movable door 104.

The spinal implant device 100 can include an upper wall 130. The upper wall 130 can span between the distal end 110 and the proximal end 114, or a portion thereof. The upper wall 130 can form an upper surface of the spinal implant device 100, or a portion thereof. The upper wall 130 can span between the distal end 110 and the movable door 104. The body structure 102 can include the upper wall 130. In some embodiments, the upper wall 130 can include one or more porous or network surfaces 132. The one or more porous or network surfaces 132 can include any of the features of the one or more porous or network surfaces described herein. The one or more porous or network surfaces 132 can be the same structure as the one or more porous or network surfaces 128.

The upper wall 130 can include a distal edge which forms the upper surface of the spinal implant device 100. The upper wall 130 can include a proximal edge which forms the upper surface of the spinal implant device 100. The upper wall 130 can include side edges which form the upper surface of the spinal implant device 100. The one or more porous or network surfaces 132 can be surrounded by the upper wall 130.

The one or more porous or network surfaces 132 can facilitate the load bearing capacity of the upper wall 130. The one or more porous or network surfaces 132 can have the same height or smaller height than another portion of the upper wall 130. The one or more porous or network surfaces 132 can have the same height or smaller height than the corresponding edges of the upper wall 130. In some embodiments, the upper wall 130 does not include one or more porous or network surfaces. In some embodiments, the upper wall 130 is open. In some embodiments, the upper wall 130 is solid. In some embodiments, the upper wall 130 is not porous. In some embodiments, the one or more porous or network surfaces 132 can form at least a portion of the upper wall 130 such as at least 30%, 35%, 40%, 45%, 50%, 55%, 60%, 65%, 70%, 75%, 80%, 85%, 90%, 95%, 100% or any range of two of the foregoing values. The upper wall 130 can surround the one or more porous or network surfaces 132. The upper wall 130 can form an edge around the one or more porous or network surfaces 132.

In some embodiments, the spinal implant device 100 can include one or more features to limit or reduce movement of the spinal implant device 100 between the vertebrae. The one or more features can allow movement in at least a first direction, such as an insertion direction. The one or more features can limit or reduce movement in at least a second direction, opposite the first direction. The one or more features can reduce the migration of the spinal implant device 100 in a direction opposite the insertion direction. The spinal implant device 100 can include a plurality of ridges 134. The plurality of ridges 134 can extend along a portion of the upper wall 130 including the edges. The plurality of ridges 134 can surround the one or more porous or network surfaces 132 of the upper wall 130. The plurality of ridges 134 can extend along a portion of the upper wall 130. The plurality of ridges 134 can extend along the proximal edge of the upper wall 130. The plurality of ridges 134 can extend along the distal edge of the upper wall 130. The plurality of ridges 134 can extend along one or both side edges of the upper wall 130.

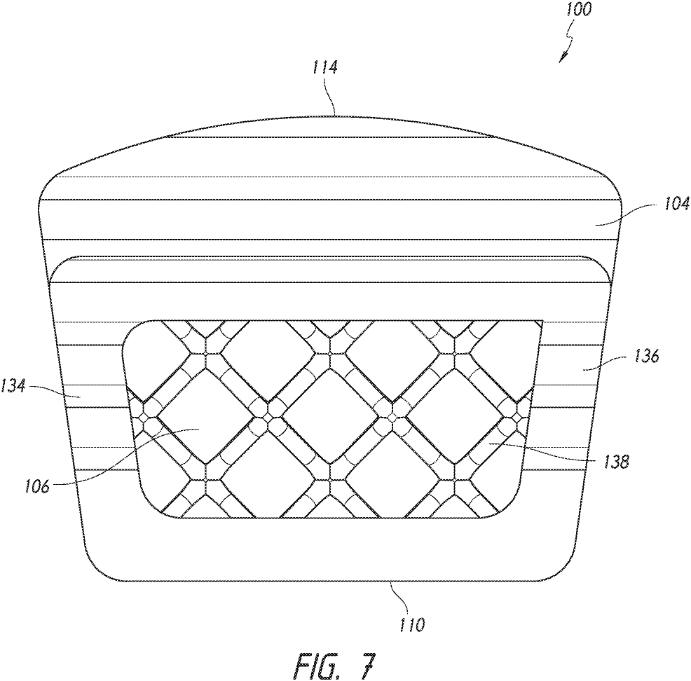

FIG. 7 is a bottom view of the spinal implant device 100. The spinal implant device 100 can include a lower wall 136. The lower wall 136 can span between the distal end 110 and the proximal end 114, or a portion thereof.

The lower wall 136 can form a lower surface of the spinal implant device 100, or a portion thereof. The lower wall 136 can span between the distal end 110 and the movable door 104. The body structure 102 can include the lower wall 136. In some embodiments, the lower wall 136 can include one or more porous or network surfaces 138. The one or more porous or network surfaces 138 can include any of the features of the one or more porous or network surfaces described herein. The one or more porous or network surfaces 138 can be the same structure as the one or more porous or network surfaces 128, 132. The one or more porous or network surfaces 138 of the lower wall 136 can have a same or similar dimensions as the one or more porous or network surfaces 132 of the upper wall 130. The one or more porous or network surfaces 132, 138 can have the same length. The one or more porous or network surfaces 132, 138 can have the same width. The one or more porous or network surfaces 132, 138 can have the same height. The one or more porous or network surfaces 132, 138 can create a passageway in a vertical direction. The one or more porous or network surfaces 132, 138 can allow ingrowth as described herein. The one or more porous or network surfaces 132, 138 can promote fusion into the cavity 106.

The lower wall 136 can include a distal edge which forms the lower surface of the spinal implant device 100. The lower wall 136 can include a proximal edge which forms the lower surface of the spinal implant device 100. The lower wall 136 can include side edges which form the lower surface of the spinal implant device 100. The one or more porous or network surfaces 138 can be surrounded by the lower wall 136.

The one or more porous or network surfaces 138 can facilitate the load bearing capacity of the lower wall 136. The one or more porous or network surfaces 138 can have the same height or smaller height than another portion of the lower wall 136. The one or more porous or network surfaces 138 can have the same height or smaller height than the corresponding edges of the lower wall 136. In some embodiments, the lower wall 136 does not include one or more porous or network surfaces. In some embodiments, the lower wall 136 is open. In some embodiments, the lower wall 136 is solid. In some embodiments, the lower wall 136 is not porous. In some embodiments, the lower wall 136 can have any features described herein. In some embodiments, the one or more porous or network surfaces 138 can form at least a portion of the lower wall 136 such as at least 30%, 35%, 40%, 45%, 50%, 55%, 60%, 65%, 70%, 75%, 80%, 85%, 90%, 95%, 100% or any range of two of the foregoing values. The lower wall 136 can surround the one or more porous or network surfaces 138. The lower wall 136 can form an edge around the one or more porous or network surfaces 138.

The plurality of ridges 134 can extend along a portion of the lower wall 136 including the edges. The plurality of ridges 134 can surround the one or more porous or network surfaces 138 of the lower wall 136. The plurality of ridges 134 can extend along a portion of the lower wall 136. The plurality of ridges 134 can extend along the proximal edge of the lower wall 136. The plurality of ridges 134 can extend along the distal edge of the lower wall 136. The plurality of ridges 134 can extend along one or both side edges of the lower wall 136. The plurality of ridges 134 can surround the one or more porous or network surfaces 132, 138. The plurality of ridges 134 can be adjacent to the porous or network surfaces 132, 138. In some embodiments, the plurality of ridges 134 can extend to a greater height than the porous or network surfaces 132, 138. In some embodiments, the plurality of ridges 134 can extend to an equal height as the porous or network surfaces 132, 138.

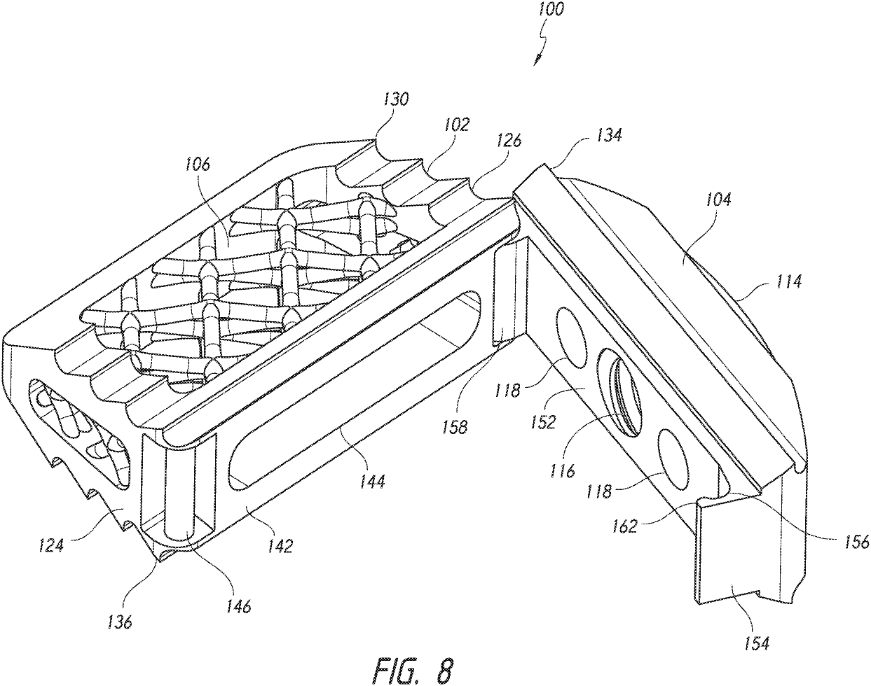

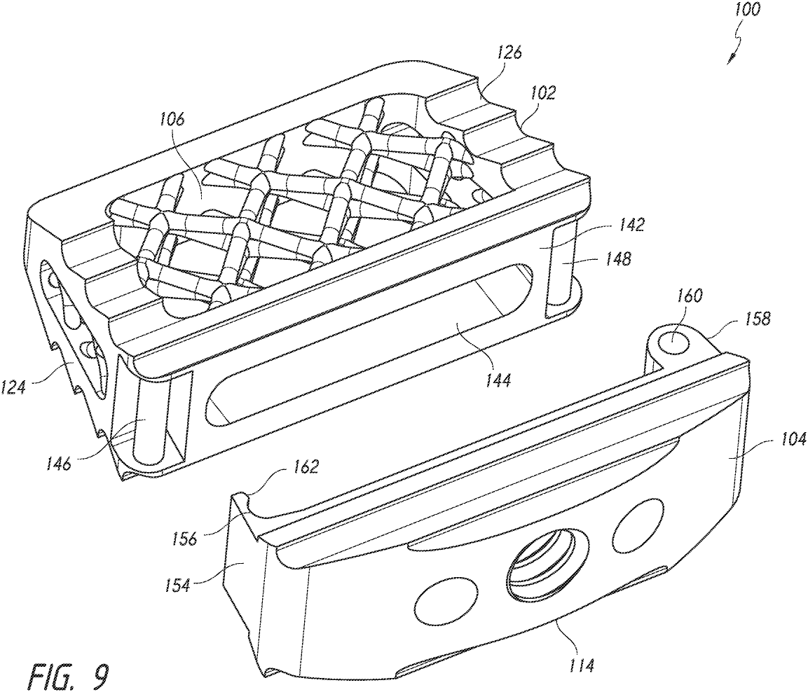

The spinal implant device 100 can include the movable door 104. FIG. 1 is a perspective view of the spinal implant device 100 with the movable door 104 closed. FIG. 8 is a perspective view of the spinal implant device 100 with the movable door 104 opened. FIG. 9 is an exploded view of the spinal implant device 100.

The movable door 104 can have one or more intermediation positons wherein the movable door 104 is partially opened. The views with the movable door 104 opened and the movable door 104 closed are for reference only. The movable door 104 can be fully closed. The movable door 104 can be partially closed. The movable door 104 can be fully opened. The movable door 104 can be partially opened. In some methods, the movable door 104 can be opened or partially opened prior to insertion. In some methods, the movable door 104 can be closed or partially closed prior to insertion. The direction of movement of the movable door 104 can allow the movable door 104 to be opened after placement between two vertebrae. The direction of movement of the movable door 104 can allow the movable door 104 to be closed after placement between two vertebrae. The movable door 104 can swing proximally to open. The movable door 104 can swing distally to close. In some embodiments, the movable door 104 can be positioned anteriorly when implanted in the patient. In some embodiments, the movable door 104 can swing anteriorly to open. In some embodiments, the movable door 104 can swing posteriorly to close. Other placements within the patient are contemplated.

The body structure 102 can include a proximal surface 142. The proximal surface 142 can be a flat surface or a generally flat surface. The proximal surface 142 can be rounded toward the sides of the spinal implant device 100. The proximal surface 142 can have a first radius of curvature extending from the first side wall 124. The proximal surface 142 can have a second radius of curvature extending from the second side wall 126. The first radius of curvature and the second radius of curvature can be the same. The first radius of curvature and the second radius of curvature can be different.

The proximal surface 142 can include an opening 144. The opening 144 can be elongate. The opening 144 can be rounded. The opening 144 can be centrally located. The opening 144 can be located along the longitudinal axis of the spinal implant device 100. The opening 144 can extend through the proximal surface 142. The opening 144 can extend into the cavity 106 of the spinal implant device 100.

The body structure 102 can include a first pin 146. The first pin 146 can be near the first side wall 124. The first pin 146 can be coupled to the body structure 102. The first pin 146 can be integrally formed with the body structure 102. The first pin 146 and the body structure 102 can be monolithically formed. The first pin 146 can be cylindrical. The first pin 146 can extend vertically. The first pin 146 can extend along the height of the body structure 102. The first pin 146 can be spaced inward from the proximal surface 142. The first pin 146 can be spaced inward from the first side wall 124. The first pin 146 can be generally aligned with the first side wall 124. The first pin 146 can be along a first side of the spinal implant device 100.

The body structure 102 can include a second pin 148. The second pin 148 can be near the second side wall 126. The second pin 148 can be coupled to the body structure 102. The second pin 148 can be integrally formed with the body structure 102. The second pin 148 and the body structure 102 can be monolithically formed. The second pin 148 can be cylindrical. The second pin 148 can extend vertically. The second pin 148 can extend along the height of the body structure 102. The second pin 148 can be spaced inward from the proximal surface 142. The second pin 148 can be spaced inward from the second side wall 126. The second pin 148 can be generally aligned with the second side wall 126. The second pin 148 can be along a second side of the spinal implant device 100. The first pin 146 and the second pin 148 can be the same or similar. The first pin 146 and the second pin 148 can have the same diameter. The first pin 146 and the second pin 148 can be disposed within the body structure 102. The first pin 146 and the second pin 148 can be diametrically opposed. The first pin 146 and the second pin 148 can be on opposite sides of the opening 144. The body structure 102 can be symmetrical. The body structure 102 can have right-left symmetry. The body structure 102 can have top-bottom symmetry. The body structure 102 can have two planes of symmetry.

The movable door 104 can include a distal surface 152. The distal surface 152 can be a flat surface or a generally flat surface. The distal surface 152 can include the opening 116. The distal surface 152 can include the one or more features 118.

The movable door 104 can include a tab 154. The tab 154 can be near the first side wall 124. The tab 154 can be near a first side of the spinal implant device 100. The tab 154 can include a passageway 156. The passageway 156 can form a portion of an arc. The passageway 156 can correspond to the outer surface of the first pin 146. The passageway 156 can engage the first pin 146. The passageway 156 can form a snap fit with the first pin 146. The tab 154 can be coupled with the movable door 104. The tab 154 can be integrally formed with the movable door 104. The tab 154 and the movable door 104 can be monolithically formed. The passageway 156 can be semi-cylindrical. The passageway 156 can extend vertically. The passageway 156 can extend along the height of the movable door 104. The tab 154 can be spaced outward from the distal surface 152. The passageway 156 can be spaced outward from the distal surface 152. The tab 154 can be along the first side of the spinal implant device 100.

The movable door 104 can include a barrel 158. The barrel 158 can be near the second side wall 126. The barrel 158 can be coupled to the movable door 104. The barrel 158 can be integrally formed with the movable door 104. The barrel 158 and the movable door 104 can be monolithically formed. The barrel 158 can be cylindrical. The barrel 158 can extend vertically. The barrel 158 can extend along the height of the movable door 104. The barrel 158 can be spaced outward from the distal surface 152. The barrel 158 can be along the second side of the spinal implant device 100. The barrel 158 can include a lumen 160. The lumen 160 can correspond to the outer surface of the second pin 148. The lumen 160 can engage the second pin 148. The lumen 160 can form a hinge with the second pin 158.

The tab 154 and the barrel 158 can be formed as features of the movable door 104. The tab 154 and the barrel 158 can be diametrically opposed. In some embodiments, the movable door 104 does not have right-left symmetry. In some embodiments, the movable door 104 has top-bottom symmetry. The movable door 104 can have one plane of symmetry.

The movable door 104 can be rotationally coupled near the second side wall 126. The movable door 104 can be rotationally coupled at the barrel 158. The movable door 104 can be rotationally coupled via the second pin 148. The movable door 104 can be rotationally coupled at the side opposite the tab 154. In other embodiments, the movable door 104 can be rotationally coupled near the first side wall 124. Any features of the spinal implant device 100 can be reversed. The features of the movable door 104 can be reversed. In some embodiments, the tab 154 can be near the second side wall 126 and the barrel 158 can be near the first side wall 124.

The spinal implant device 100 can include an articulation. The spinal implant device 100 can include a movable j oint. The movable j oint can include the second pin 148 and the barrel 158. The movable joint can include the second pin 148 that extends through the lumen 160 of the barrel 158. The second pin 148 and the barrel 158 can be designed for rotation. The second pin 148 can be a pivot pin. The second pin 148 can be an axle. The articulation can include the second pin 148 and the lumen 160. The articulation can be vertical. The articulation can extend along the height of the spinal implant device 100, or a portion thereof. The second pin 148 and the lumen 160 can be aligned in the direction of the height of the spinal implant device 100. The articulation can be perpendicular or generally perpendicular to the longitudinal axis of the spinal implant device 100. The second pin 148 and the lumen 160 can be perpendicular or generally perpendicular to a longitudinal axis of the spinal implant device 100. The second pin 148 and the lumen 160 can be along a longitudinal axis of the patient when implanted. In some embodiments, the movable joint can include one or more pins (e.g., one pin, two pins, three pins, four pins, five pins, or six pins). The articulation can include two or more separate pins. The separate pins can be aligned along a single rotational axis. The movable joint can be formed in a number of ways including one or more pins. The spinal implant device 100 can include a single rotational axis.

The movable joint including the second pin 148 and the barrel 158 can be 3D printed, as described herein. The articulation can be formed within the body structure 102. The articulation can be formed within the movable door 104. The second pin 148 can be captive within the barrel 158. The second pin 148 can be prevented from removal. The second pin 148 can form an axis of rotation. The second pin 148 can be a hinge pin. The second pin 148 can be integrally formed to extend through the barrel 158. The barrel 158 can rotate relative to the second pin 148. The barrel 158 and the second pin 148 can form a hinge. The movable door 104 can rotate relative to the body structure 102.

The barrel 158 can include the lumen 160 configured to engage the second pin 148. The lumen 160 can extend along the height of the spinal implant device 100, or a portion thereof. In some embodiments, the articulation can be offset from the longitudinal axis of the spinal implant device 100. The articulation can be aligned along one of the opposing side walls 124, 126. The articulation can be aligned with the second side wall 126 of the spinal implant device 100.

The articulation can extend between the upper surface and the lower surface. The articulation can extend through the barrel 158 of the movable door 104. The articulation can extend within the recessed portion of the second side wall 126. The articulation can extend along the second side wall 126. The articulation can be any structure about which the movable door 104 can rotate. The barrel 158 can include the lumen 160 to receive the second pin 148. The lumen 160 can extend entirely through the barrel 158, or a portion thereof. The lumen 160 can extend entirely through the movable door 104, or a portion thereof. The articulation can be perpendicular to the longitudinal axis of the spinal implant device 100.

In some embodiments, the movable joint can couple the movable door 104 to the body structure 102. The movable joint can couple two structures of the spinal implant device 100. The movable joint can allow for a range of motion of the movable door. The movable joint can allow for at least 30 degrees of rotation, 45 degrees of rotation, 60 degrees of rotation, 75 degrees of rotation, 80 degrees of rotation, 85 degrees of rotation, 90 degrees of rotation, 95 degrees of rotation, 100 degrees of rotation, 120 degrees of rotation, or any range of two of the foregoing values. The movable joint can allow for rotational motion of the movable door 104. The movable joint can allow the movable door 104 to open and close about an axis of rotation. The axis of the movable j oint can be perpendicular to the longitudinal axis of the spinal implant device 100.

In some embodiments, the spinal implant device 100 can include the tab 154. The tab 154 can be located along the first side wall 124. The tab 154 can include the passageway 156. The passageway 156 can have a recessed shape. The tab 154 can include a recessed portion that extends inward. The first side wall 124 can provide a visual cue to the location of the tab 154. The tab 154 can facilitate opening the movable door 104. The tab 154 can facilitate closing the movable door 104. The tab 154 can be located near the proximal end 114. The user can apply a force to the movable door 104 from the side of the movable door 104. The force can be applied along the first side of the movable door 104. The tab 154 can facilitate the swinging of the movable door 104. The tab 154 can facilitate the rotating of the movable door 104. The tab 154 can facilitate swinging the movable door 104 relative to the second pin 148. The movable door 104 swings as the barrel 158 rotates relative to the second pin 148. The tab 154 can facilitate the application of force to disengage the passageway 156 of the movable door 104 relative to the first pin 146. The tab 154 can facilitate the application of force to engage the passageway 156 of the movable door 104 with the first pin 146. The tab 154 and the first pin 146 can disengage to open to movable door 104. The tab 154 and the first pin 146 can engage to close the movable door 104.

In some embodiments, the tab 154 overhangs the first side wall 124. The tab 154 can form a lip relative to the first side wall 124. The tab 154 and the first pin 146 can form a frictional fit. The passageway 156 can include a catch 162. The catch 162 can limit or prevent further rotation of the moveable door 104. The catch 162 can abut the first pin 146 to limit rotation. The catch 162 of the movable door 104 can contact the first pin 146 along the first side wall 124 when the movable door 104 is fully closed. The first pin 146 can interact with the catch 162. The catch 162 can prevent further rotation of the movable door 146 in one direction when the catch 162 is engaged. In some embodiments, the catch 162 of the movable door 104 and the first pin 146 of the body structure 102 interlock together. The catch 162 can provide tactile feedback that the movable door 104 is closed.

In some embodiments, the distal surface 152 of the movable door 104 can contact the proximal surface 142 of the body structure 102 along the entire height of the movable door 104, or a portion thereof, when the movable door 104 is closed. In some embodiments, the distal surface 152 of the movable door 104 can contact the proximal surface 142 of the body structure 102 along the entire width of the movable door 104, or a portion thereof, when the movable door 104 is closed.

The movable door 104 and the body structure 102 can have any complementary shape. The body structure 102 can form a support surface that mirrors the shape of the movable door 104. In some embodiments, the movable door 104 contacts the proximal surface 142 of the body structure 102 when the movable door 140 is closed. In some embodiments, the movable door 104 is spaced apart from the proximal surface 142 of the body structure 102 when the movable door 140 is closed. In some embodiments, the movable door 104 contacts the first side wall 124 when the movable door 104 is closed. In some embodiments, the movable door 104 contacts the first pin 146 when the movable door 104 is closed.

The movable door 104 and the body structure 102 can have corresponding surfaces. The body structure 102 can form the recessed portion to accommodate the barrel 158 of the movable door 104. The body structure 102 can form the recessed portion to accommodate the tab 154 of the movable door 104.

The movable door 104 can align with the body structure 102. The first side wall 124 and the tab 154 can form the first side surface of the spinal implant device 100. The movable door 104 can align with the body structure 102 to form the first side surface of the spinal implant device 100. The movable door 104 and the body structure 102 together can form the first side surface of the spinal implant device 100. The first side surface of the spinal implant device 100 can include the first side wall 124. The first side surface of the spinal implant device 100 can include the movable door 104. The first side surface of the spinal implant device 100 can include the tab 154.

The second side wall 126 and the barrel 158 can form the second side surface of the spinal implant device 100. The movable door 104 can align with the body structure 102 to form the second side surface of the spinal implant device 100. The movable door 104 and the body structure 102 together can form the second side surface of the spinal implant device 100. The second side surface of the spinal implant device 100 can include the second side wall 126. The second side surface of the spinal implant device 100 can include the movable door 104. The second side surface of the spinal implant device 100 can include the barrel 158.

The upper wall 130 and the movable door 104 can form the upper surface of the spinal implant device 100. The movable door 104 can align with the body structure 102 to form the upper surface of the spinal implant device 100. The movable door 104 and the body structure 102 together can form the upper surface of the spinal implant device 100. The upper surface of the spinal implant device 100 can include the upper wall 130. The upper surface of the spinal implant device 100 can include the movable door 104.

The lower wall 136 and the movable door 104 can form the lower surface of the spinal implant device 100. The movable door 104 can align with the body structure 102 to form the lower surface of the spinal implant device 100. The movable door 104 and the body structure 102 together can form the lower surface of the spinal implant device 100. The lower surface of the spinal implant device 100 can include the lower wall 136. The lower surface of the spinal implant device 100 can include the movable door 104.

In some embodiments, the upper wall 130 is adjacent to the movable door 104 when the movable door 104 is closed. In some embodiments, the lower wall 136 is adjacent to the movable door 104 when the movable door 104 is closed. In some embodiments, the first side wall 124 is adjacent to the movable door 104 when the movable door 104 is closed. In some embodiments, the second side wall 126 is adjacent to the movable door 104 when the movable door 104 is closed.

The movable door 140 can form a portion of the top surface of the spinal implant device 100. The movable door 140 can form a portion of the lower surface of the spinal implant device 100. The movable door 140 can form a portion of the first side surface of the spinal implant device 100. The movable door 140 can form a portion of the second side surface of the spinal implant device 100.

The movable door 140 can extend across the entire width of the spinal implant device 100. The movable door 140 can extend across the entire height of the spinal implant device 100. The movable door 140 can extend across a portion of the length of the spinal implant device 100.

The movable door 104 can form a minority of the length of the spinal implant device 100. The movable door 104 can form at least a portion of the length such as at least 5%, 10%, 15%, 20%, 25%, 30%, 35%, 40%, 45%, 50% or any range of two of the foregoing values. The movable door 104 can include the proximal end 114. In some embodiments, the movable door 104 extends between the side walls 124, 126. The movable door 104 can be sized to engage the first pin 146. The upper wall 130 can form a majority of the upper surface of the spinal implant device 100. The upper wall 130 can form at least a portion of the upper surface such as at least 50%, 55%, 60%, 65%, 70%, 75%, 80%, 85%, 90%, 95%, 100% or any range of two of the foregoing values. In some embodiments, the movable door 104 and the upper wall 130 can provide a load supporting surface. In some methods, the movable door 104 and the upper wall 130 can be configured to contact a vertebral end plate of a superior vertebra. In some embodiments, the porous or network surfaces 132 can form at least a portion of the upper surface of the spinal implant device 100 such as at least 30%, 35%, 40%, 45%, 50%, 55%, 60%, 65%, 70%, 75%, 80%, 85%, 90%, 95%, 100% or any range of two of the foregoing values.

The lower wall 136 can form a majority of the lower surface of the spinal implant device 100. The lower wall 136 can form at least a portion of the lower surface such as at least 50%, 55%, 60%, 65%, 70%, 75%, 80%, 85%, 90%, 95%, 100% or any range of two of the foregoing values. In some embodiments, the movable door 104 and the lower wall 136 can provide a load supporting surface. In some methods, the movable door 104 and the lower wall 136 can be configured to contact a vertebral end plate of an inferior vertebra. In some embodiments, the porous or network surfaces 138 can form at least a portion of the lower surface of the spinal implant device 100 such as at least 30%, 35%, 40%, 45%, 50%, 55%, 60%, 65%, 70%, 75%, 80%, 85%, 90%, 95%, 100% or any range of two of the foregoing values.

In some embodiments, the moveable door 104 can pivot from a generally closed position relative to the body structure 102. The moveable door 104 can be aligned along the width of the spinal implant device 100. The movable door 104 can be transverse to the longitudinal axis of the spinal implant device 100. In some embodiments, the moveable door 104 can pivot to a generally open position. The moveable door 104 can be aligned along the second side wall 126 of the spinal implant device 100. The movable door 104 can be aligned with or parallel to the longitudinal axis of the spinal implant device 100. The movable door 104 can swing in plane. The movable door 104 can be generally aligned with the upper wall 130 and the lower wall 136 when opened. The movable door 104 can be generally aligned with the upper wall 130 and the lower wall 136 when closed. The moveable door 104 can swing relative to a vertical axis of rotation. The moveable door 104 can be aligned with the second side wall 126 when opened. In some embodiments, the moveable door 140 can pivot to any angle such as 15 degrees, 30 degrees, 45 degrees, 60 degrees, 75 degrees, 90 degrees, 105 degrees, 120 degrees, 135 degrees, 150 degrees, 165 degrees, 180 degrees, or any range of two of the foregoing values.

The movable door 104 can match the taper of the upper wall 130. The movable door 104 can match the taper of the lower wall 136. The movable door 140 can match the lordosis angle. The movable door 104 can include a plurality of ridges 134. The plurality of ridges 134 can extend along a portion of the movable door 104 including the edges. The plurality of ridges 134 can extend along a portion of the upper surface of the movable door 104. The plurality of ridges 134 can extend along a portion of the lower surface of the movable door 104. The plurality of ridges 134 can extend along a portion of the upper surface including movable door 104 and the upper wall 130. The plurality of ridges 134 can extend along a portion of the lower surface including the movable door 104 and the lower wall 136.

The spinal implant device 100 can include the cavity 106. In some embodiments, the movable door 104 can define a proximal inner surface of the cavity 118. The movable door 104 can close the opening 144 of the proximal surface 142 of the body structure 102. The distal surface 152 can include a generally flat inner surface. The movable door 104 can be thin walled to increase the volume of the cavity 106. In some embodiments, the distal end 110 can define a distal inner surface of the cavity 106. The distal end 110 can include a flattened inner surface. The distal end 110 can be hollow. The distal end 110 can be thin walled to increase the volume of the cavity 106. In some embodiments, the two opposing side walls 124, 126 can define the side inner surfaces of the cavity 106. The spinal implant device 100 can define a lordosis angle between the side walls 124, 126. The lordosis angle can shape the cavity 106. In some embodiments, the upper wall 130 can define the upper inner surface of the cavity 106. In some embodiments, the lower wall 136 can define the lower inner surface of the cavity 106. The cavity 106 can be a defined space within the spinal implant device 100.

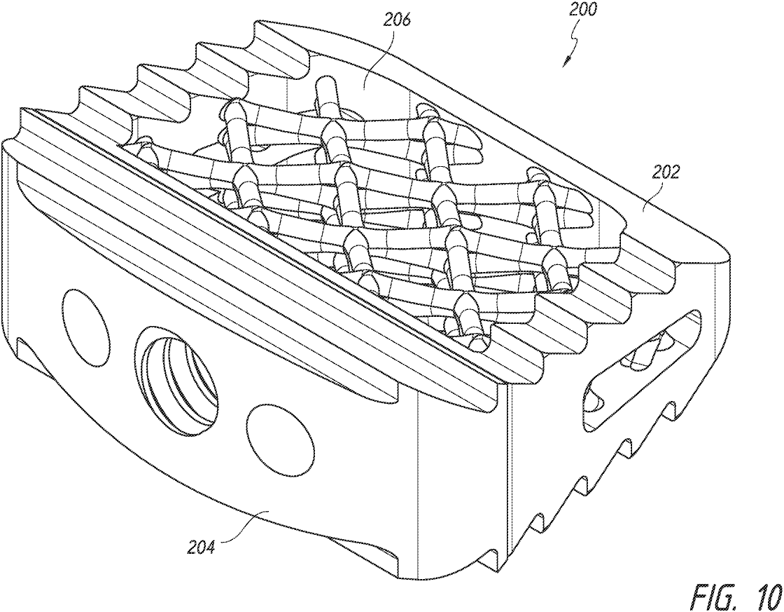

FIG. 10 illustrates a perspective view of a spinal implant device 200. The spinal implant device 200 can be placed between adjacent vertebrae. The spinal implant device 200 can include a body structure 202. The body structure 202 can form a majority of the spinal implant device 200. The body structure 202 can form a majority of the length of the spinal implant device 200. The spinal implant device 200 can include a movable door 204. The movable door 204 can form a portion of the spinal implant device 200. The movable door 204 can form a portion of the length of the spinal implant device 200. The spinal implant device 200 can include a cavity 206. The movable door 204 can couple to the body structure 202. The movable door 204 can rotate relative to the body structure 202. The movable door 204 can allow the cavity 206 to be opened to be packed with material. The movable door 204 can allow the cavity 206 to be closed. The spinal implant device 200 can be designed for vertebral fusion as described herein. The spinal implant device 200 can include any feature described herein. The spinal implant device 200 can be configured for insertion between any two vertebrae. The spinal implant device 200 can be configured for insertion between two cervical vertebrae. The spinal implant device 200 can be configured for insertion between two lumbar vertebrae. The spinal implant device 200 can be configured for insertion between two thoracic vertebrae. The spinal implant device 200 can be configured for any approach. The spinal implant device 200 can be configured for an anterior approach. The spinal implant device 100 can be configured for a posterior approach.

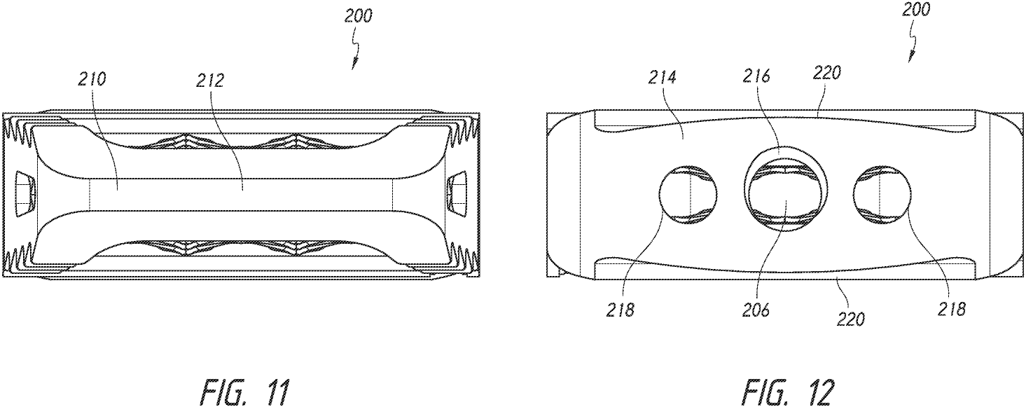

FIG. 11 is a distal view of the spinal implant device 200. The spinal implant device 200 can include a distal end 210. In some methods of use, the distal end 210 can be the insertion end. In some embodiments, the distal end 210 forms a wedge shape. The distal end 210 can slope downward along an upper surface. The distal end 210 can slope upward along a lower surface. The distal end 204 can form a leading edge 212. The leading edge 212 can be flattened. The leading edge 212 can be rounded. The distal end 210 can include one or more corners or edges. The distal end 210 can be rounded toward the sides of the spinal implant device 200. The distal end 210 can have a first curved edge extending from a first side surface. The distal end 210 can have a second curved edge extending from a second side surface. The curved edges can be the same. The curved edges can be different. The body structure 202 can include the distal end 210.

In some embodiments, the spinal implant device 200 can include an inclination toward one side of the spinal implant device 200. The spinal implant device 200 can have a lordosis angle. The spinal implant device 200 can have a lordosis angle to correspond to the natural orientation of the vertebral endplates. In some embodiments, the spinal implant device 200 does not include a lordosis angle. The lordosis angle can be zero or an angle such as 0°, 1°, 2°, 3°, 4°, 5°, 6°, 7 °, 8°, 9°, 10°, 11°, 12°, 13°, 14°, 15°, 16°, 17°, 18°, 19°, 20°, between 0° and 5°, between 0° and 6°, between 0° and 7°, between 3° and 9°, between 5° and 7°, between 6° and 12°, between 8° and 16°, between 10° and 14°, between 12° and 14°, between 16° and 20°, approximately 6°, approximately 12°, approximately 18°, or any range of two of the foregoing values. In some embodiments, the distal end 210 is tapered to one side by the lordosis angle as described herein. In some embodiments, the distal end 210 can taper consistent with the lordosis angle of the spinal implant device 200.

FIG. 12 is a proximal view of the spinal implant device 200. The spinal implant device 200 can include a proximal end 214. In some embodiments, the proximal end 214 is tapered to one side by the lordosis angle as described herein. In some embodiments, the proximal end 214 can taper consistent with the lordosis angle of the spinal implant device 200. The proximal end 214 can be a convex surface. The proximal end 214 can bow outward. The proximal end 214 can be rounded toward the sides of the spinal implant device 200. The proximal end 214 can have a first curved edge extending from the first side surface. The distal end 220 can have a second curved edge from the second side surface. The first curved edge and the second curved edge can be the same. The first curved edge and the second curved edge can be different. The movable door 204 can include the proximal end 214.

The proximal end 214 can engage with an insertion tool. The proximal end 214 can include an opening 216. The opening 216 can be circular. The opening 216 can be through the wall of the proximal end 214. The opening 216 can be threaded to engage a threaded end of the insertion tool. The opening 216 can be centrally located between sides on the movable door 204. The opening 216 can be centrally located along the height of the movable door 204. The opening 216 can be located at a neutral center of the spinal implant device 200. The opening 216 can be located along the longitudinal axis of the spinal implant device 200. The opening 216 can extend entirely through the proximal end 214. The opening 216 can extend entirely through the movable door 204. The opening 216 can extend into the cavity 206 of the spinal implant device 200.

The spinal implant device 200 can include one or more features 218 to facilitate placement of the spinal implant device 200. The proximal end 214 can include one or more features 218. The feature 218 can be circular. The feature 218 can have a smaller diameter than the opening 216. The feature 218 can be through the wall of the proximal end 214. The feature 218 can be non-threaded to engage a non-threaded end of the insertion tool. The feature 218 can be centrally located along the height of the movable door 204. The feature 218 can be offset from the longitudinal axis of the spinal implant device 200. The feature 218 can extend entirely through the proximal end 214. The feature 218 can extend entirely through the movable door 204. The feature 218 can extend into the cavity 206 of the spinal implant device 200. In the illustrated embodiment, the one or more features 218 include two openings. The one or more features 218 can be diametrically opposed relative to the opening 216. The one or more features 218 can be equally spaced relative to the opening 216.

The opening 216 and the one or more features 218 can facilitate placement of the spinal implant device 200. The opening 216 and the one or more features 218 can prevent relative translational movement between the spinal implant device 200 and the insertion tool. The opening 216 and the one or more features 218 can prevent relative rotational movement between the spinal implant device 200 and the insertion tool. The proximal end 214 can include one or more undercuts 220. The top surface of the proximal end 214 can include the undercut 220. The lower surface of the proximal end 214 can include the undercut 220. The one or more undercuts 220 can facilitate engagement with the insertion tool.

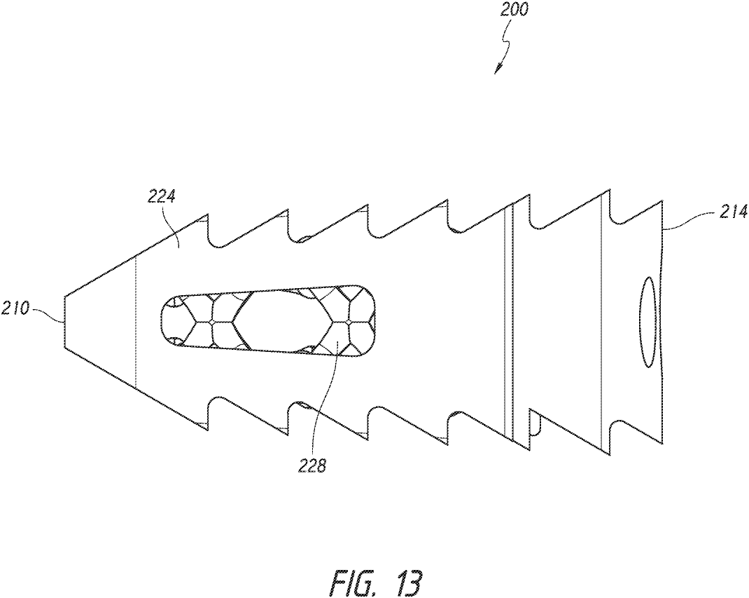

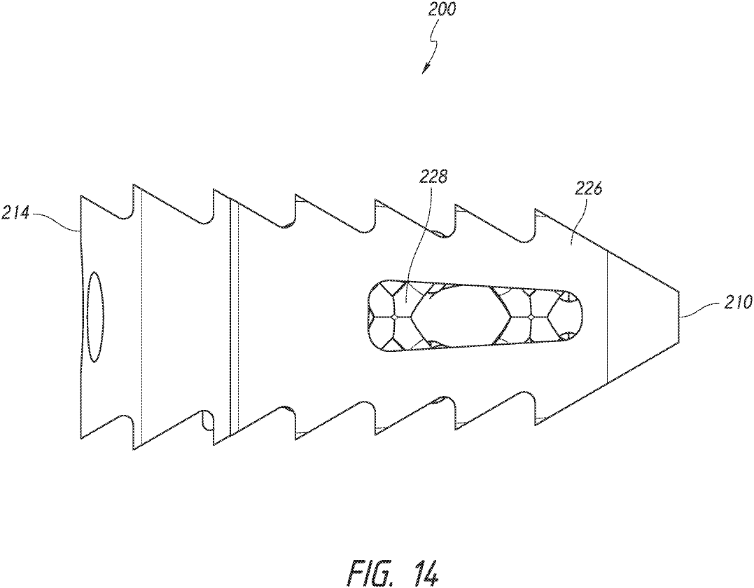

FIG. 13 is a side view of the spinal implant device 200. FIG. 14 is another side view of the spinal implant device 200. In some embodiments, the spinal implant device 200 can have a length measured between the distal end 210 and the proximal end 214. The spinal implant device 200 can include two side walls including a first side wall 224 and a second side wall 226. FIG. 13 illustrates the first side wall 224. FIG. 14 illustrates the second side wall 226. The first side wall 224 and the second side wall 226 can include the same or similar dimensions. The first side wall 224 and the second side wall 226 can include the same or similar features. The first side wall 224 and the second side wall 226 can be the same or similar. The first side wall 224 and the second side wall 226 can be identical. The first side wall 224 and the second side wall 226 can include different dimensions. The first side wall 224 and the second side wall 226 can include different features. The first side wall 224 and the second side wall 226 can include different heights. The first side wall 224 and the second side wall 226 can include different surface features.

The first side wall 224 can extend along the length of the spinal implant device 200, or a portion thereof. The second side wall 226 can extend along the length of the spinal implant device 200, or a portion thereof. The first side wall 224 and the second side wall 226 can be opposing side walls. In some embodiments, the two side walls 224, 226 are skewed along at least a portion of the length of the spinal implant device 200. some embodiments, the two side walls 224, 226 taper toward the distal end 210. In some embodiments, the two side walls 224, 226 are spaced apart along at least a portion of the length of the spinal implant device 200.

The spinal implant device 200 can have a length or depth. The two side walls 224, 226 can define at least a portion of the length or depth of the spinal implant device 200. The movable door 204 can define at least a portion of the length or depth of the spinal implant device 200. The spinal implant device 200 can define a range of lengths. The spinal implant device 200 can have a maximum length of 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 20 mm, 21 mm, 22 mm, 23 mm, 24 mm, 25 mm, 26 mm, 27 mm, 28 mm, 29 mm, 20 mm, 30 mm, 31 mm, 32 mm, 33 mm, 34 mm, 35 mm, between 6 mm and 10 mm, between 10 mm and 15 mm, between 15 mm and 20 mm, between 23 mm and 27 mm, between 25 mm and 29 mm, between 27 mm and 31 mm, between 29 mm and 33 mm, or any range of two of the foregoing values. The length of the second side wall 226 can be less than the length of the first side wall 224. The length of the first side wall 224 can be less than the length of the second side wall 226. The first side wall 224 and the second side wall 226 can have the same or similar length.

The two side walls 224, 226 can define the width of the spinal implant device 200. The width can vary along the length. The two side walls 224, 226 can define a range of widths along at least a portion of the length of the spinal implant device 200. The maximum width as measured between the two side walls 224, 226 can be 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, 25 mm, 25.5 mm, 26 mm, 26.5 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, 35 mm, 35.5 mm, 36 mm, 36.5 mm, 37 mm, 37.5 mm, 38 mm, 38.5 mm, 39 mm, 39.5 mm, 40 mm, 40.5 mm, 41 mm, 41.5 mm, 42 mm, 42.5 mm, 43 mm, 43.5 mm, 44 mm, 44.5 mm, 45 mm, between 10 mm and 15 mm, between 14 mm and 16 mm, between 16 mm and 18 mm, between 17 mm and 20 mm, between 20 mm and 22 mm, between 28 mm and 32 mm, between 35 mm and 39 mm, between 39 mm and 43 mm, between 30 mm and 37 mm, between 30 mm and 41 mm, or any range of two of the foregoing values.

The two side walls 224, 226 can extend along the height of the spinal implant device 200. The two side walls 224, 226 can define the height of the spinal implant device 200, or a portion thereof. The height can vary based on the lordosis angle. The height can taper toward the distal end 210. The two side walls 224, 226 can define a range of heights. The first side wall 224 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The second side wall 226 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The height of the second side wall 226 can be the same or similar to the height of the first side wall 224. The height of the second side wall 226 can be less than the height of the first side wall 224. The lordosis angle can taper downward from the first side wall 224 toward the second side wall 226. The height of the first side wall 224 can be less than the height of the second side wall 226. The lordosis angle can taper downward from the second side wall 226 toward the first side wall 224.

In some embodiments, the first side wall 224 can include one or more porous or network surfaces 228. In some embodiments, the second side wall 226 can include one or more porous or network surfaces 228. The one or more porous or network surfaces 228 can have any feature of the one or more porous or network surfaces described herein.

The porous or network surfaces 228 can be a matrix. The porous or network surfaces 228 can be square or rectangular. The porous or network surfaces 228 can be planar. The porous or network surfaces 228 can be non-planar. The porous or network surfaces 228 can be two-dimensional. The porous or network surfaces 228 can be three-dimensional. The porous or network surfaces 228 can include rows extending along one plane. The porous or network surfaces 228 can include rows extending along another plane. The porous or network surfaces 228 can form diamond pores. The porous or network surfaces 228 can form any shaped pores including polygonal or rounded pores. The porous or network surfaces 228 can include any structure to promote bony fusion. The porous or network surfaces 228 can include any mesh structure. The porous or network surfaces 228 can be a three-dimensional shaped surface. The porous or network surfaces 228 can include any shaped openings or pores. The porous or network surfaces 228 can be cross-linked. The porous or network surfaces 228 can include one or more layers. The porous or network surfaces 228 can comprise a lattice unit cell. The porous or network surfaces 228 can include intersecting struts. In some embodiments, the openings of porous or network surfaces 228 can cover a majority of the surface of the porous or network surfaces 228. In some embodiments, the struts or structure of porous or network surfaces 228 can cover a minority of the surface of the porous or network surfaces 228.

The first side wall 224 can include one or more sections of porous or network surfaces 228. The first side wall 224 can include one section of porous or network surfaces 228. The section of the porous or network surfaces 228 can be generally elongate. The porous or network surfaces 228 can extend along the length of the first side wall 224. In some embodiments, the struts or structure of porous or network surfaces 228 can cover a majority of the first side wall 224. The first side wall 224 can be generally linear. The sections of the porous or network surfaces 228 of the first side wall 224 can mirror the linear shape of first side wall 224. The porous or network surfaces 228 can facilitate the load bearing capacity of the first side wall 224. In some embodiments, the spinal implant device 200 does not include the porous or network surfaces 228 on the first side wall 224. In some embodiments, the first side wall 224 comprises one or more openings. In some embodiments, the first side wall 224 is open. In some embodiments, the first side wall 224 is closed. In some embodiments, the first side wall 224 is solid. In some embodiments, the first side wall 224 is not porous.

The one or more porous or network surfaces 228 can have the same width or smaller width than another portion of the first side wall 224. The first side wall 224 can include an opening supported by thicker edges. The porous or network surfaces 228 can be disposed within the opening. The first side wall 224 can surround the porous or network surfaces 228. The porous or network surfaces 228 can facilitate the load bearing capacity of the first side wall 224. In some embodiments, the first side wall 224 can have any features described herein.

The second side wall 226 can include one or more sections of porous or network surfaces 228. The second side wall 226 can include one section of porous or network surfaces 228. The section of the porous or network surfaces 228 can be generally elongate. The porous or network surfaces 228 can extend along the length of the second side wall 226. In some embodiments, the struts or structure of porous or network surfaces 228 can cover a majority of the second side wall 226. The second side wall 226 can be generally linear. The sections of the porous or network surfaces 228 of the second side wall 226 can mirror the linear shape of second side wall 226. The porous or network surfaces 228 can facilitate the load bearing capacity of the second side wall 226. In some embodiments, the spinal implant device 200 does not include the porous or network surfaces 228 on the second side wall 226. In some embodiments, the second side wall 226 comprises one or more openings. In some embodiments, the second side wall 226 is open. In some embodiments, the second side wall 226 is closed. In some embodiments, the second side wall 226 is solid. In some embodiments, the second side wall 226 is not porous.

The one or more porous or network surfaces 228 can have the same width or smaller width than another portion of the second side wall 226. The second side wall 226 can include an opening supported by thicker edges. The porous or network surfaces 228 can be disposed within the opening. The second side wall 226 can surround the porous or network surfaces 228. The porous or network surfaces 228 can facilitate the load bearing capacity of the second side wall 226. In some embodiments, the second side wall 226 can have any features described herein.

The one or more distinct sections of porous or network surfaces 228 can have any feature of the one or more porous or network surfaces described herein. The porous or network surfaces 228 can be the same matrix on the first side wall 224 and the second side wall 226. The porous or network surfaces 228 of the first side wall 224 and the second side wall 226 can have the same length or generally the same length. The porous or network surfaces 228 of the first side wall 224 and the second side wall 226 can have the same width or generally the same width. The porous or network surfaces 228 of the first side wall 224 and the second side wall 226 can have the same height or generally the same height.

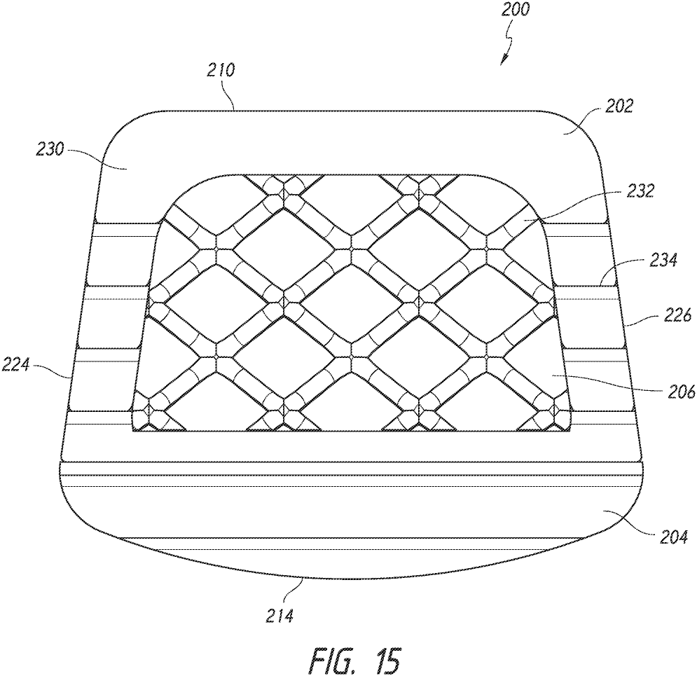

FIG. 15 is a top view of the spinal implant device 200. The two opposing side walls 224, 226 can extend between the distal end 210 and the proximal end 214. The separation of the two opposing side walls 224, 226 define the width of the spinal implant device 200. In some embodiments, the two opposing side walls 224, 226 are separated by a varying width along the length of the two opposing side walls 224, 226. In some embodiments, the two opposing side walls 224, 226 generally taper toward the distal end 210. In some embodiments, the two opposing side walls 224, 226 are generally at an angle to each other. In some embodiments, the two opposing side walls 224, 226 gradually taper. In some embodiments, the two opposing side walls 224, 226 have a smaller width near the distal end 210 than near the proximal end 214. In some embodiments, the first side wall 224 is straight or generally straight along a majority of the length of the first side wall 224. In some embodiments, the second side wall 226 is straight or generally straight along a majority of the length of the second side wall 226. In some embodiments, the two opposing side walls 224, 226 are slightly curved along a majority of the length of the two opposing side walls 224, 226. The two opposing side walls 224, 226 can bow outward. The two opposing side walls 224, 226 can bow inward.

The spinal implant device 200 can include an upper wall 230. The upper wall 230 can span between the distal end 210 and the proximal end 214, or a portion thereof. The upper wall 230 can span between the distal end 210 and the movable door 204. In some embodiments, the upper wall 230 can include one or more porous or network surfaces 232. The one or more porous or network surfaces 232 can include any of the features of the one or more porous or network surfaces described herein. The one or more porous or network surfaces 232 can be the same structure as the one or more porous or network surfaces 228. The struts and the pores of the one or more porous or network surfaces 232 of the upper wall 230 can have a same or similar dimensions as the one or more porous or network surfaces 228 of the opposing side walls 224, 226.

The one or more porous or network surfaces 232 can be surrounded by the upper wall 230. The upper wall 230 can include an opening. The one or more porous or network surfaces 232 can be disposed within the opening. The upper wall 230 can include a distal edge which forms the upper surface of the spinal implant device 200. The upper wall 230 can include a proximal edge which forms the upper surface of the spinal implant device 200. The upper wall 230 can include side edges which form the upper surface of the spinal implant device 200. The upper wall 230 can facilitate the load bearing capacity of the spinal implant device 200. The one or more porous or network surfaces 232 can facilitate the load bearing capacity of the spinal implant device 200. The one or more porous or network surfaces 232 can have the same height or smaller height than another portion of the upper wall 230. In some embodiments, the upper wall 230 does not include one or more porous or network surfaces 232. In some embodiments, the upper wall 230 comprises one or more openings. In some embodiments, the upper wall 230 is open. In some embodiments, the upper wall 230 is solid or not porous. In some embodiments, the upper wall 230 can have any features described herein.

In some embodiments, the spinal implant device 200 can include one or more features to increase traction of the spinal implant device 200 between the vertebrae. The one or more features can allow movement in an insertion direction The one or more features can limit or reduce movement in a retraction direction. The one or more features can reduce the back-out of the spinal implant device 200. The spinal implant device 200 can include a plurality of ridges 234. The plurality of ridges 234 can be wedge shaped. The plurality of ridges 234 can extend along a portion of the upper wall 230. The plurality of ridges 234 can surround the one or more porous or network surfaces 232 of the upper wall 230.

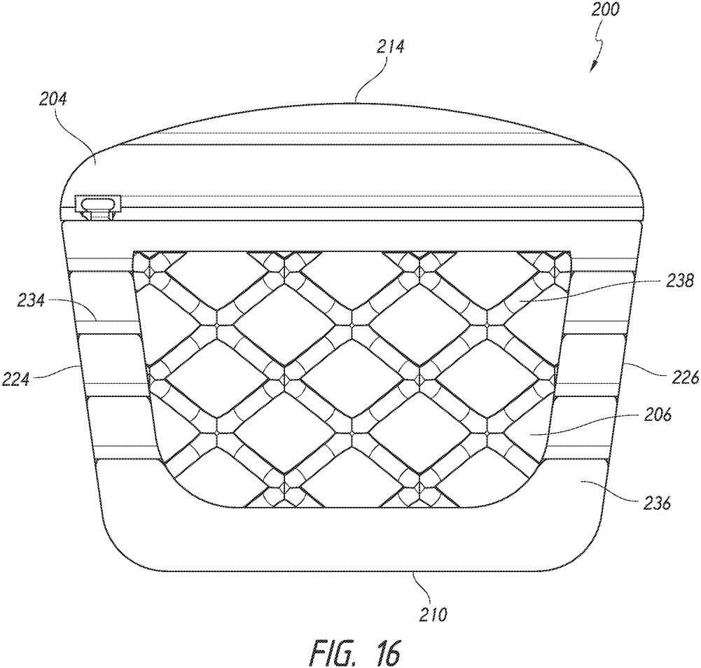

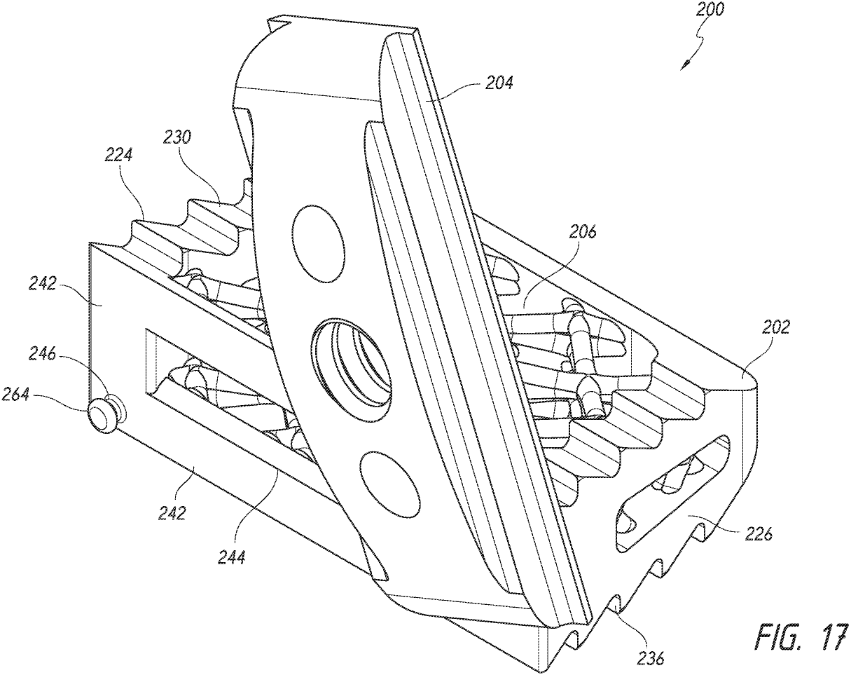

FIG. 16 is a bottom view of the spinal implant device 200. The spinal implant device 200 can include a lower wall 236. The lower wall 236 can span between the distal end 210 and the proximal end 214, or a portion thereof. The lower wall 236 can span between the distal end 210 and the movable door 204. In some embodiments, the lower wall 236 can include one or more porous or network surfaces 238. The one or more porous or network surfaces 238 can include any of the features of the one or more porous or network surfaces described herein. The one or more porous or network surfaces 238 can be the same structure as the one or more porous or network surfaces 228, 232. The one or more porous or network surfaces 238 can be diametrically opposed to the one or more porous or network surfaces 232. The one or more porous or network surfaces 232, 238 can form a through lumen through the spinal implant device 200. The one or more porous or network surfaces 232, 238 can allow fusion in the vertical direction. The one or more porous or network surfaces 232, 238 can allow fusion between adjacent vertebral endplates. The one or more porous or network surfaces 232, 238 can form a passageway between adjacent vertebrae. The one or more porous or network surfaces 232, 238 can form a passageway into the cavity 206. The section of one or more porous or network surfaces 238 of the lower wall 236 can have a same or similar dimensions as the section of one or more porous or network surfaces 232 of the upper wall 230. The one or more porous or network surfaces 232, 238 can have the same length. The one or more porous or network surfaces 232, 238 can have the same width. The one or more porous or network surfaces 232, 238 can have the same height. The one or more porous or network surfaces 232, 238 can allow bony ingrowth to promote fusion. The one or more porous or network surfaces 232, 238 can be generally aligned in the vertical direction.

The one or more porous or network surfaces 238 can be surrounded by the lower wall 236. The lower wall 236 can include one or more edges around the one or more porous or network surfaces 238. The lower wall 236 can include a distal edge which forms the lower surface of the spinal implant device 200. The lower wall 236 can include a proximal edge which forms the lower surface of the spinal implant device 200. The lower wall 236 can include side edges which form the lower surface of the spinal implant device 200. The lower wall 236 can facilitate the load bearing capacity of the spinal implant device 200. The one or more porous or network surfaces 238 can facilitate the load bearing capacity of the spinal implant device 200. The one or more porous or network surfaces 238 can have the same height or smaller height than the corresponding edges of the lower wall 236. In some embodiments, the lower wall 236 does not include one or more porous or network surfaces. In some embodiments, the lower wall 236 comprises one or more openings. In some embodiments, the lower wall 236 is solid. In some embodiments, the lower wall 236 can have any features described herein.

The plurality of ridges 234 can extend along a portion of the lower wall 236. The plurality of ridges 234 can extend along the proximal edge of the lower wall 236. The plurality of ridges 234 can extend along the distal edge of the lower wall 236. The plurality of ridges 234 can extend along the side edges of the lower wall 236.

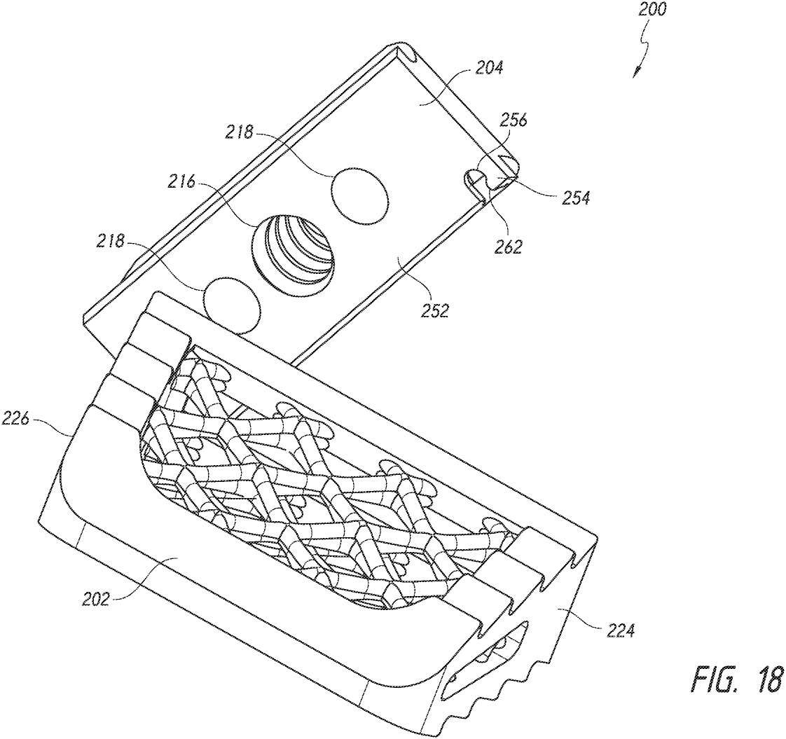

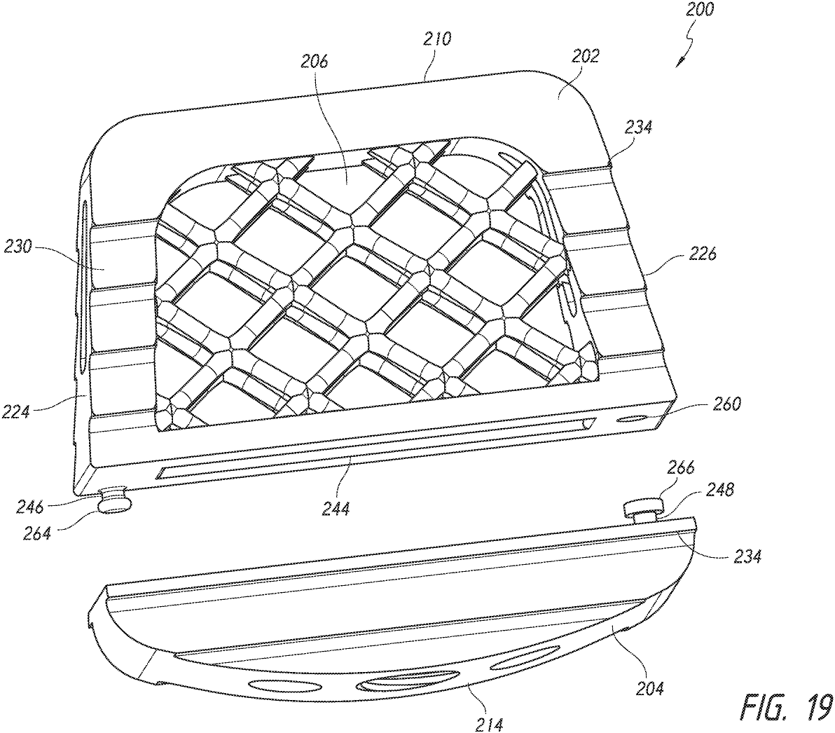

The spinal implant device 200 can include the movable door 204. FIG. 10 is a perspective view of the spinal implant device 200 with the movable door 204 closed. FIG. 17 is a perspective view of the spinal implant device 200 with the movable door 204 opened. FIG. 18 is a perspective view of the spinal implant device 200 with the movable door 204 opened. FIG. 19 is an exploded view of the spinal implant device 200.

The movable door 204 can have one or more intermediation positons wherein the movable door 204 is partially opened or partially closed. The movable door 204 can be fully closed or fully opened. In some methods, the movable door 204 can be opened or partially opened prior to insertion. In some methods, the movable door 204 can be closed or partially closed prior to insertion. The movable door 204 can be configured to be opened or closed prior to insertion. In some methods, the movable door 204 does not open after insertion. The direction of movement of the movable door 204 may not allow the movable door 204 to be opened after placement between two vertebrae. The direction of movement of the movable door 204 may not allow the movable door 204 to be closed after placement between two vertebrae.

The body structure 202 can include a proximal surface 242. The proximal surface 242 can be a flat surface or a generally flat surface. The proximal surface 242 can be shaped to abut the movable door 204. The proximal surface 242 can have any complementary shape to the movable door 204.

The proximal surface 242 can include an opening 244. The opening 244 can be elongate. The opening 244 can be rectangular. The opening 216 can be through the wall of the proximal surface 242. The opening 244 can be centrally located. The opening 244 can extend into the cavity 206 of the spinal implant device 200. The opening 244 can be the same or similar shape as the cavity 206.

The body structure 202 can include a first pin 246. The first pin 246 can be near the first side wall 224. The first pin 246 can be coupled to the body structure 202. The first pin 246 can be integrally formed with the body structure 202. The first pin 246 and the body structure 202 can be monolithically formed. The first pin 246 can be cylindrical. The first pin 246 can include one or more diameters. The first pin 246 can include a flange 264. The first pin 246 can extend horizontally. The first pin 246 can extend along the length of the body structure 202. The first pin 246 can extend outward from the proximal surface 242. The first pin 246 can be spaced inward from the first side wall 224. The first pin 246 can be spaced inward from the lower wall 236. The first pin 246 can be generally aligned with the first side wall 224. The first pin 246 can be along a first side of the spinal implant device 200. The first pin 246 can be closer to the lower wall 236 than the upper wall 230. The first pin 246 can function as a catch as described herein.

The moveable door 204 can include a second pin 248. The second pin 248 can be coupled to the moveable door 204. The second pin 248 can be integrally formed with the moveable door 204. The second pin 248 and the moveable door 204 can be monolithically formed. The second pin 248 can be cylindrical. The second pin 248 can include one or more diameters. The second pin 248 can include a flange 266. The second pin 248 can extend horizontally. The second pin 248 can be spaced outward from the moveable door 204. The second pin 248 can function as a hinge as described herein.

The movable door 204 can include a distal surface 252. The distal surface 252 can be a flat surface or a generally flat surface. The distal surface 252 can include the opening 216. The distal surface 252 can include the one or more features 218. The distal surface 252 can include the second pin 248.

The movable door 204 can include a tab 254. The distal surface 252 can include the tab 254. The tab 254 can be near the first side wall 224. The tab 254 can be near a first side of the spinal implant device 200. The tab 254 can include a passageway 256. The passageway 256 can have a recessed shape. The passageway 256 can form a portion of an arc. The tab 254 can include a recessed portion that extends inward. The passageway 256 can correspond to the outer surface of the first pin 246. The passageway 256 can engage the first pin 246. The passageway 256 can received the flange 264 of the first pin 246. The passageway 256 can form a stop with the first pin 246. The tab 254 can be coupled with the movable door 204. The tab 254 can be integrally formed with the movable door 204. The tab 254 and the movable door 204 can be monolithically formed. The passageway 256 can be cylindrical. The passageway 256 can extend from the lower surface of the movable door 204. The passageway 256 can extend inward from the movable door 204. The tab 204 can be aligned with the distal surface 252. The passageway 256 can be spaced inward from the distal surface 252. The tab 254 can be generally aligned with the first side wall 224. The tab 254 can be along a first side of the spinal implant device 200.

The body structure 202 can include a lumen 260. The lumen 260 can receive the second pin 248. The lumen 260 can extend horizontally. The lumen 260 can be generally aligned with the second side wall 226. The lumen 260 can be along a second side of the spinal implant device 200. The lumen 260 can be shaped to correspond to the outer surface of the second pin 258. The lumen 260 can engage the second pin 258. The lumen 260 can form a hinge with the second pin 258. The lumen 260 can be along the length of the spinal implant device 200. The second pin 248 can include the flange 266. The flange 266 can retain the second pin 248 within the lumen 260. The lumen 260 can receive the flange 266 of the second pin 248. The second pin 248 can be generally aligned with the second side wall 226. The second pin 248 can be along the second side of the spinal implant device 200. The second pin 248 can extend along a portion of the length of the body structure 202.

The first pin 246 and the second pin 248 can be different. The first pin 246 and the second pin 248 can have different shapes. The first pin 246 and the second pin 248 can have different functions. The first pin 246 and the second pin 248 can have the same or similar diameter. The first pin 246 can extend from the body structure 202. The second pin 248 can extend from the movable door 204. The second pin 248 can be disposed within the body structure 202. The second pin 248 can be captive. The second pin 248 can couple the movable door 204 to the body structure 202.

The first pin 246 and the second pin 248 can be on opposite sides of the spinal implant device 200. The first pin 246 and the second pin 248 can be diametrically opposed relative to the opening 244. The first pin 246 and the second pin 248 can be offset. The first pin 246 can be closer to the lower wall 236 than the second pin 248 is to the lower wall 236. The second pin 248 can be equally spaced between the upper wall 230 and the lower wall 236. The body structure 202 can be non-symmetrical. The body structure 202 can have right-left non-symmetry. The body structure 202 can have top-bottom non-symmetry. The movable door 204 can be non-symmetrical. The movable door 204 can have right-left non-symmetry. The movable door 204 can have top-bottom non-symmetry.

The movable door 204 can be rotationally coupled near the second side wall 226. The movable door 204 can be rotationally coupled at the side opposite the tab 254. In other embodiments, the movable door 204 can be rotationally coupled near the first side wall 224. Any features of the body structure 202 can be reversed. Any features of the movable door 204 can be reversed.

In some embodiments, the movable door 204 can include an articulation. The articulation can include the second pin 248 and the lumen 260. The articulation can be horizontal. The articulation can extend along the length of the spinal implant device 200, or a portion thereof. The second pin 248 and the lumen 260 can be aligned in the direction of the length of the spinal implant device 200. The articulation can be parallel or generally parallel to the longitudinal axis of the spinal implant device 200. The second pin 248 and the lumen 260 can be parallel or generally parallel to the longitudinal axis of the spinal implant device 200. The second pin 248 and the lumen 260 can be along an anterior-posterior axis of the patient when implanted. The articulation including the second pin 248 and the lumen 260 can be 3D printed. The articulation can be formed within the body structure 202. The articulation can extend from the movable door 204. The second pin 248 can be captive within the body structure 202. The second pin 248 can be prevented from removal. The second pin 248 can form an axis of rotation. The second pin 248 can be a hinge pin. The second pin 248 can extend through the body structure 202. The second pin 248 can rotate relative to the body structure 202. The lumen 260 and the second pin 248 can form a hinge.

The body structure 202 can include the lumen 260 configured to engage the second pin 248. The lumen 260 can extend along a portion of the length of the spinal implant device 200. In some embodiments, the articulation can be offset from the longitudinal axis of the spinal implant device 200. The articulation can be aligned along one of the opposing side walls. The articulation 262 can be aligned with the second side wall 226 of the spinal implant device 200.

The articulation can extend between the distal end 210 and the proximal end 214. The articulation can extend from the movable door 204. The articulation can extend within the lumen 260 of body structure 202. The articulation can extend along the second side wall 226. The articulation can be any structure about which the movable door 204 can rotate.

In some embodiments, the spinal implant device 200 can include a movable joint. The movable joint can couple the movable door 204 and the body structure 202. The movable joint can allow for a range of motion of the movable door 204. The movable joint can allow for at least 30 degrees of rotation, 45 degrees of rotation, 60 degrees of rotation, 75 degrees of rotation, 80 degrees of rotation, 85 degrees of rotation, 90 degrees of rotation, 95 degrees of rotation, 200 degrees of rotation, 220 degrees of rotation, or any range of two of the foregoing values. The movable joint can allow for rotational motion of the movable door 204. The movable joint can allow for rotational motion relative to the second pin 248. The movable joint can allow the movable door 204 to open and close about an axis of rotation of the second pin 248. The axis of the movable joint can be parallel or generally parallel to the longitudinal axis of the spinal implant device 200.

The user can apply a force to the movable door 204 from the lower surface of the movable door 204. The force can be applied underneath the movable door 204. The movable door 204 swings relative to the second pin 248. The tab 254 can function as a stop relative to the first pin 246. The tab 254 can rest against the first pin 246 under the influence of gravity. In some embodiments, the distal surface 252 of the movable door 204 can contact the proximal surface 242 of the body structure 202, when the movable door 204 is closed. In some embodiments, the movable door 204 is spaced apart from the proximal surface 242 of the body structure 202 when the movable door 204 is closed.

The movable door 204 and the body structure 202 together can form the upper surface of the spinal implant device 200. The upper surface of the spinal implant device 200 can include the upper wall 230 and the movable door 204. The movable door 204 and the body structure 202 together can form the lower surface of the spinal implant device 200. The lower surface of the spinal implant device 200 can include the lower wall 236 and the movable door 204. The movable door 204 and the body structure 202 together can form the first side surface of the spinal implant device 200. The first side surface of the spinal implant device 200 can include the first side wall 224 and the movable door 204. The movable door 204 and the body structure 202 together can form the second side surface of the spinal implant device 200. The second side surface of the spinal implant device 200 can include the second side wall 226 and the movable door 204.

In some embodiments, the upper wall 230 is flush with the movable door 204 when the movable door 204 is closed. In some embodiments, the lower wall 236 is flush with the movable door 204 when the movable door 204 is closed. In some embodiments, the first side wall 224 is flush with the movable door 204 when the movable door 204 is closed. In some embodiments, the second side wall 226 is flush with the movable door 204 when the movable door 204 is closed.

The movable door 204 can swing clockwise to open. The movable door 204 can swing open by movement of the second pin 248 relative to the lumen 260. The movable door 204 can swing counterclockwise to close. The movable door 204 can swing close by movement of the second pin 248 relative to the lumen 260. The movable door 204 can close when the passageway 256 engages the first pin 246. The passageway 256 can include a catch 262. The catch 262 can limit or prevent further rotation of the moveable door 204. The catch 262 can abut the first pin 246 to limit rotation. The catch 262 of the movable door 204 can contact the first pin 246 along the first side wall 224 when the movable door 204 is fully closed. The first pin 246 can interact with the catch 262. The catch 262 can prevent further rotation of the movable door 246 in one direction when the catch 262 is engaged. In some embodiments, the catch 262 of the movable door 204 and the first pin 246 of the body structure 202 interlock together. The catch 262 can provide tactile feedback that the movable door 204 is closed.

In some embodiments, the moveable door 204 can pivot from a generally closed position relative to the body structure 202. The moveable door 204 can extend across the width of the spinal implant device 200. The moveable door 204 can be generally horizontal. In some embodiments, the moveable door 204 can pivot to an open position. The moveable door 204 can rotate out of plane. The moveable door 204 can have a horizontal axis of rotation. The moveable door 204 can be generally aligned with the upper wall 230 and the lower wall 236 when closed. The moveable door 204 can be generally skewed relative to the upper wall 230 and the lower wall 236 when opened.

The movable door 204 can correspond to the taper of the upper wall 230. The movable door 204 can correspond to the taper of the lower wall 236. The movable door 240 can include the lordosis angle. The movable door 204 can include a plurality of ridges 234. The plurality of ridges 234 can extend along a portion of the top surface of the movable door 204. The plurality of ridges 234 can extend along a portion of the lower surface of the movable door 204.

The spinal implant device 200 can include the cavity 206. In some embodiments, the movable door 204 can define an inner surface of the cavity 206. The movable door 204 can close the opening 244 of the proximal surface 242 of the body structure 202. The distal surface 252 can define an inner surface of the cavity 206. In some embodiments, the distal end 210 can define an inner surface of the cavity 206. In some embodiments, the two opposing side walls 224, 226 can define inner surfaces of the cavity 206. The spinal implant device 200 can define a lordosis angle between the side walls 224, 226. The lordosis angle can shape the cavity 206. In some embodiments, the upper wall 230 can define an inner surface of the cavity 206. In some embodiments, the lower wall 236 can define an inner surface of the cavity 206. The cavity 206 can be a defined space within the spinal implant device 200.

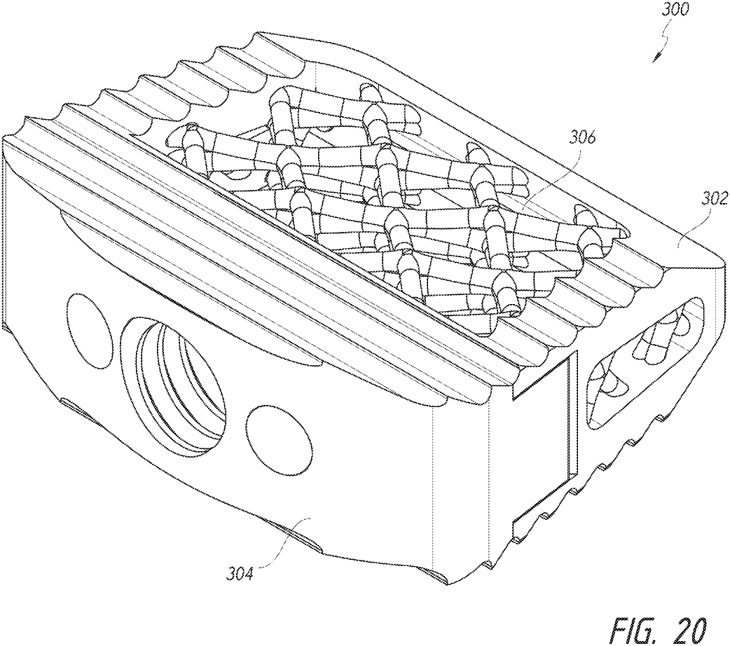

FIG. 20 illustrates a perspective view of a spinal implant device 300. The spinal implant device 300 can include a body structure 302. The spinal implant device 300 can include a movable door 304. The movable door 304 can couple to the body structure 302, as described herein. The body structure 302 and the movable door 304 can define a cavity 306. The movable door 304 can allow the cavity 306 to be filled with material. The spinal implant device 300 can include any feature described herein. The spinal implant device 300 can be configured for insertion between any two vertebrae. The spinal implant device 300 can be configured for insertion between two cervical vertebrae. The spinal implant device 300 can be configured for insertion between two lumbar vertebrae. The spinal implant device 300 can be configured for insertion between two thoracic vertebrae. The spinal implant device 300 can be configured for any approach. The spinal implant device 300 can be configured for an anterior approach. The spinal implant device 300 can be configured for a posterior approach.

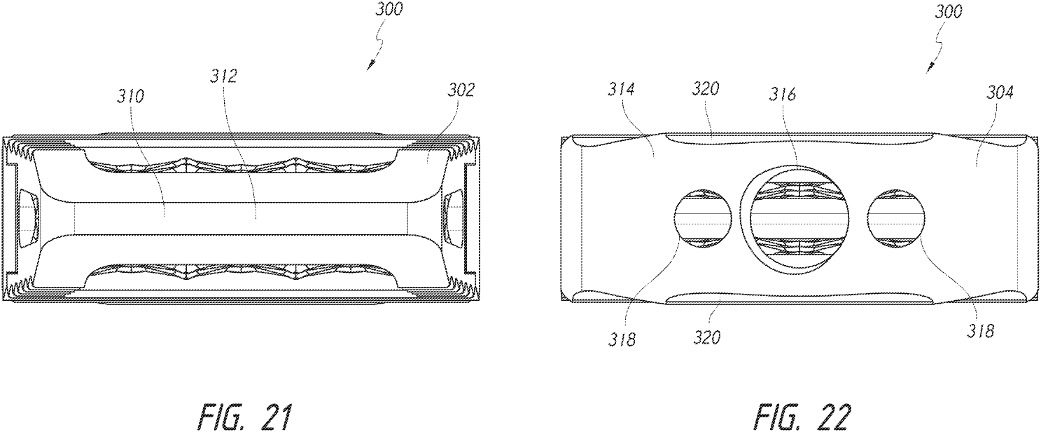

FIG. 21 is a distal view of the spinal implant device 300. The spinal implant device 300 can include a distal end 310. In some embodiments, the distal end 310 forms a wedge shape. The distal end 304 can form a leading edge 312. The leading edge 312 can be flattened or blunt. The leading edge 312 can be curved. The body structure 302 can form the distal end 310.

FIG. 22 is a proximal view of the spinal implant device 300. The spinal implant device 300 can include a proximal end 314. In some embodiments, the proximal end 314 can be a flat shape. The movable door 304 can form the proximal end 314.

The spinal implant device 300 may or may not have a lordosis angle. The lordosis angle can be zero or an angle greater than zero. The lordosis angle can be an angle such as 0°, 1°, 2°, 3°, 4°, 5°, 6°, 7 °, 8°, 9°, 10°, 11°, 12°, 13°, 14°, 15°, 16°,17° 18°, 19°, 20°, between 0° and 5°, between 0° and 6°, between 0° and 7°, between 3° and 9°, between 5° and 7°, between 6° and 12°, between 8° and 16°, between 10° and 14°, between 12° and 14°, between 16° and 20°, approximately 6°, approximately 12°, approximately 18°, or any range of two of the foregoing values. The distal end 310 and the proximal end 314 can follow the lordosis angle.

The proximal end 314 can engage with an insertion tool. In some embodiments, the proximal end 314 can include an opening 316. The opening 316 can engage an end of the insertion tool. In some embodiments, the proximal end 314 can include one or more features 318. The one or more features 318 can engage an end of the insertion tool. In some embodiments, the proximal end 314 can include one or more undercuts 320. The opening 316, the one or more features 318, and/or the undercut 320 can prevent translational movement between the spinal implant device 300 and the insertion tool. The opening 316, the one or more features 318, and/or the undercut 320 can prevent rotational movement between the spinal implant device 300 and the insertion tool.

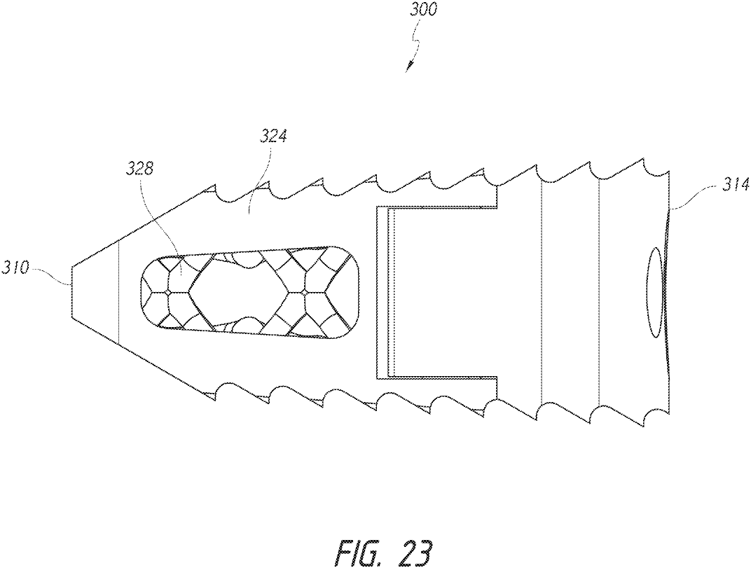

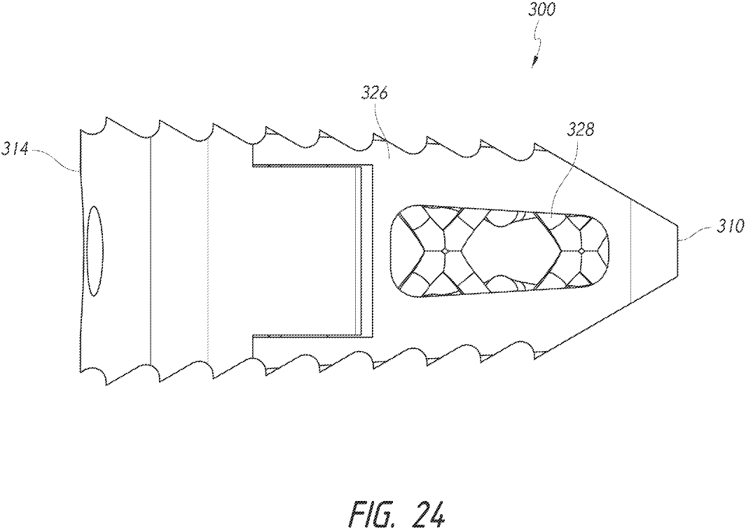

FIG. 23 is a side view of the spinal implant device 300. FIG. 24 is another side view of the spinal implant device 300. The spinal implant device 300 can include two side walls including a first side wall 324 and a second side wall 326. FIG. 23 illustrates the first side wall 324. FIG. 24 illustrates the second side wall 326. The first side wall 324 and the second side wall 326 can include the same or similar features. The first side wall 324 and the second side wall 326 can include different features.

The two side walls 324, 326 are spaced apart along at least a portion of the length of the spinal implant device 300. In some embodiments, the two side walls 324, 326 can taper along at least a portion of the length of the spinal implant device 300. The two side walls 324, 326 can define a range of widths of the spinal implant device 300. The two side walls 324, 326 can extend along the height of the spinal implant device 300. The height of one side wall can be less than the height of the other side wall. The lordosis angle can taper toward one side wall.

The spinal implant device 300 can have a length or depth. The two side walls 324, 326 can define at least a portion of the length or depth of the spinal implant device 300. The movable door 304 can define at least a portion of the length or depth of the spinal implant device 300. The spinal implant device 300 can define a range of lengths. The spinal implant device 300 can have a maximum length of 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 20 mm, 21 mm, 22 mm, 23 mm, 24 mm, 25 mm, 26 mm, 27 mm, 28 mm, 29 mm, 20 mm, 30 mm, 31 mm, 32 mm, 33 mm, 34 mm, 35 mm, between 6 mm and 10 mm, between 10 mm and 15 mm, between 15 mm and 20 mm, between 23 mm and 27 mm, between 25 mm and 29 mm, between 27 mm and 31 mm, between 29 mm and 33 mm, or any range of two of the foregoing values. The length of the second side wall 326 can be less than the length of the first side wall 324. The length of the first side wall 324 can be less than the length of the second side wall 326. The first side wall 324 and the second side wall 326 can have the same or similar length.

The maximum width as measured between the two side walls 324, 326 can be 10 mm, 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, 25 mm, 25.5 mm, 26 mm, 26.5 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, 35 mm, 35.5 mm, 36 mm, 36.5 mm, 37 mm, 37.5 mm, 38 mm, 38.5 mm, 39 mm, 39.5 mm, 40 mm, 40.5 mm, 41 mm, 41.5 mm, 42 mm, 42.5 mm, 43 mm, 43.5 mm, 44 mm, 44.5 mm, 45 mm, between 10 mm and 15 mm, between 14 mm and 16 mm, between 16 mm and 18 mm, between 17 mm and 20 mm, between 20 mm and 22 mm, between 28 mm and 32 mm, between 35 mm and 39 mm, between 39 mm and 43 mm, between 30 mm and 37 mm, between 30 mm and 41 mm, or any range of two of the foregoing values.

The first side wall 324 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values. The second side wall 326 can have a maximum height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, or any range of two of the foregoing values.

In some embodiments, the first side wall 324 can include one or more porous or network surfaces 328. In some embodiments, the second side wall 326 can include one or more porous or network surfaces 328. The one or more porous or network surfaces 328 can have any feature of the one or more porous or network surfaces described herein. The porous or network surfaces 328 can be any shaped surface described herein. The porous or network surfaces 328 can include inter-linked struts that form pores. The porous or network surfaces 328 can include a three-dimensional shape to promote fusion.

The first side wall 324 can include one or more distinct sections of porous or network surfaces 328. The sections of the porous or network surfaces 328 of the first side wall 324 can mirror the curvature or linear orientation of the first side wall 324. The porous or network surfaces 328 can increase the load bearing capacity of the first side wall 324. In some embodiments, the first side wall 324 can have any features described herein.

The second side wall 326 can include one or more distinct sections of porous or network surfaces 328. The sections of the porous or network surfaces 328 of the second side wall 326 can mirror the curvature or linear orientation of second side wall 326. The porous or network surfaces 328 can increase the load bearing capacity of the second side wall 326. In some embodiments, the second side wall 326 can have any features described herein. The porous or network surfaces 328 can have any feature of the one or more porous or network surfaces described herein.

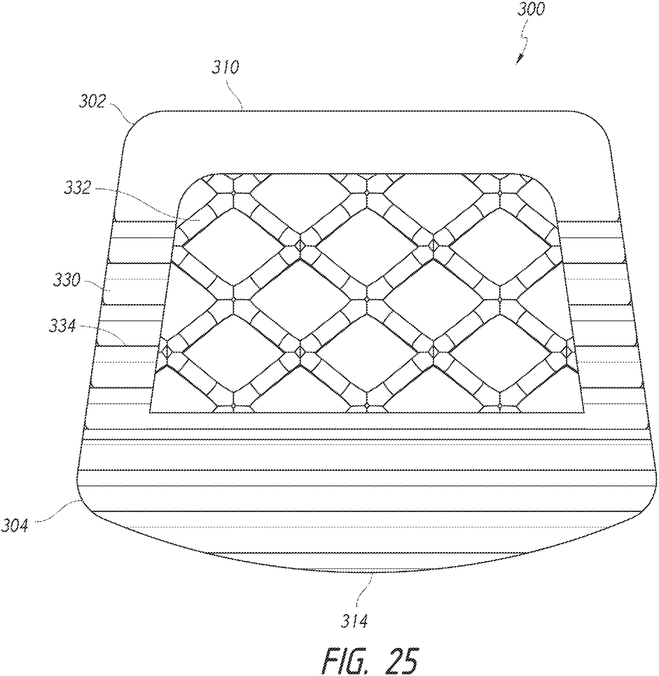

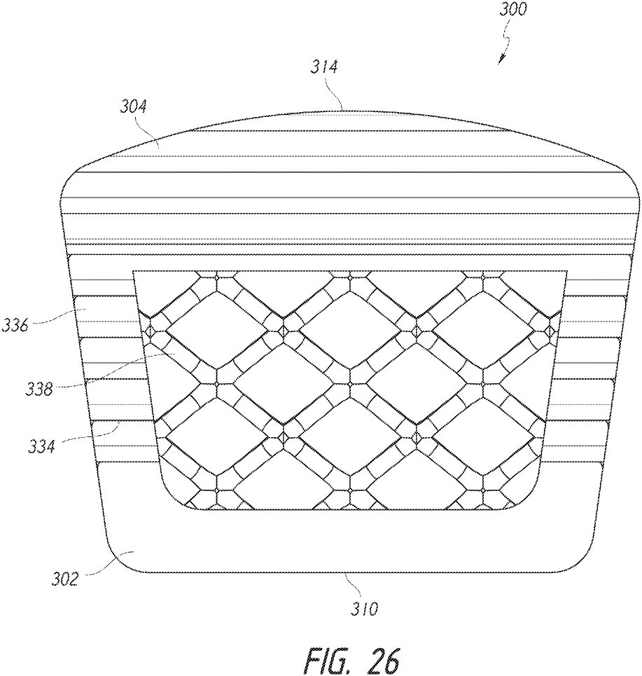

FIG. 25 is a top view of the spinal implant device 300. The spinal implant device 300 can include an upper wall 330. The upper wall 330 can span between the distal end 310 and the movable door 304. In some embodiments, the upper wall 330 can include one or more porous or network surfaces 332. The one or more porous or network surfaces 332 can include any of the features of the one or more porous or network surfaces described herein. The one or more porous or network surfaces 332 can be the same structure as the one or more porous or network surfaces 328.

The upper wall 330 can include a distal edge which forms the upper surface of the spinal implant device 300. The upper wall 330 can include a proximal edge which forms the upper surface of the spinal implant device 300. The upper wall 330 can include side edges which form the upper surface of the spinal implant device 300. The one or more porous or network surfaces 332 can be surrounded by the upper wall 330. The one or more porous or network surfaces 332 can increase the load bearing capacity of the upper wall 330. The spinal implant device 300 can include a plurality of ridges 334. The plurality of ridges 334 can extend along a portion of the upper wall 330.

FIG. 26 is a bottom view of the spinal implant device 300. The spinal implant device 300 can include a lower wall 336. The lower wall 336 can span between the distal end 310 and the movable door 304. In some embodiments, the lower wall 336 can include one or more porous or network surfaces 338. The one or more porous or network surfaces 338 can include any of the features of the one or more porous or network surfaces described herein. The one or more porous or network surfaces 338 can be the same structure as the one or more porous or network surfaces 328, 332.

The lower wall 336 can include a distal edge which forms the lower surface of the spinal implant device 300. The lower wall 336 can include a proximal edge which forms the lower surface of the spinal implant device 300. The lower wall 336 can include side edges which form the lower surface of the spinal implant device 300. The one or more porous or network surfaces 338 can be surrounded by the lower wall 336. The one or more porous or network surfaces 338 can increase the load bearing capacity of the lower wall 336. The plurality of ridges 334 can extend along a portion of the lower wall 336.

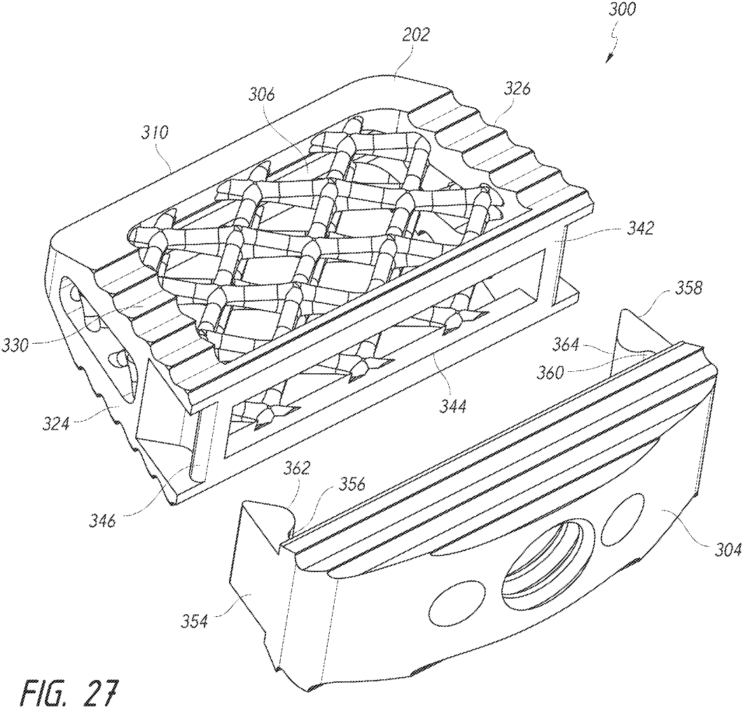

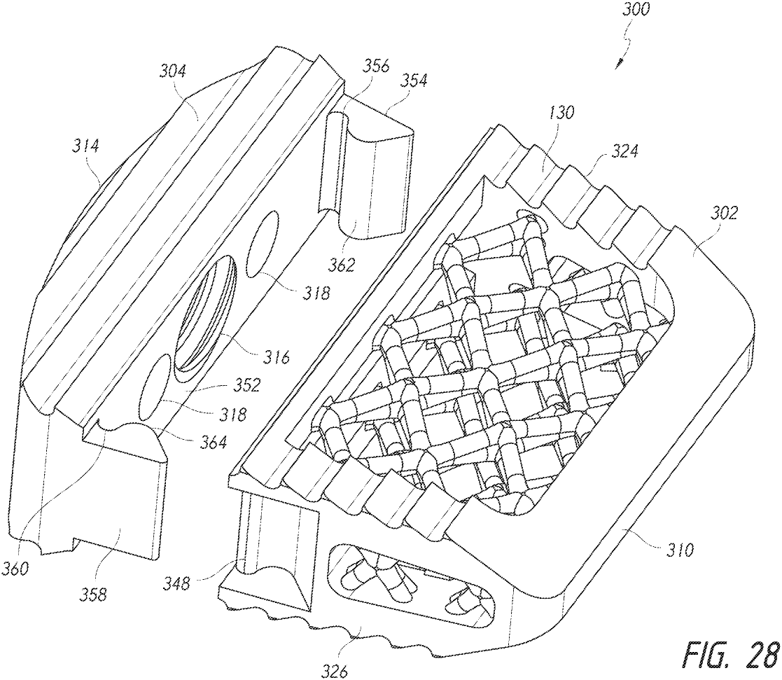

The spinal implant device 300 can include a movable door 304. FIG. 20 is a perspective view of the spinal implant device 300 with the movable door 304 closed. FIG. 27 is a perspective view of the spinal implant device 300 with the movable door 304 opened. FIG. 28 is another perspective view of the spinal implant device 300 with the movable door 304 opened.

The movable door 304 can have one or more intermediation positons wherein the movable door 304 is partially opened or partially closed. In some methods, the movable door 304 can be opened or partially opened prior to insertion. In some methods, the movable door 304 can be closed or partially closed prior to insertion. The direction of movement of the movable door 304 can allow the movable door 304 to be opened after placement between two vertebrae. The direction of movement of the movable door 304 can allow the movable door 304 to be closed after placement between two vertebrae.

The body structure 302 can include a proximal surface 342. The proximal surface 342 can be a flat surface or generally flat surface. The proximal surface 342 can be rounded toward the sides of the spinal implant device 300. The proximal surface 342 can have one or more radii of curvature extending from a first side surface. The proximal surface 342 can have one or more radii of curvature extending from a second side surface. The first radius of curvature and the second radius of curvature can be the same. The first radius of curvature and the second radius of curvature can be different.

The proximal surface 342 can include an opening 344. The opening 344 can be elongate. The opening 344 can extend into the cavity 306 of the spinal implant device 300. The opening 344 can allow the cavity 306 to be packed.

The body structure 302 can include a first protrusion 346. The first protrusion 346 can be near the first side wall 324. The first protrusion 346 can be coupled to the body structure 302. The first protrusion 346 can be integrally formed with the body structure 302. The first protrusion 346 and the body structure 302 can be monolithically formed. The first protrusion 346 can be semi-cylindrical. The first protrusion 346 can extend vertically. The first protrusion 346 can extend along the height of the body structure 302. The first protrusion 346 can be spaced inward from the proximal surface 342. The first protrusion 346 can be spaced inward from the first side wall 324. The first protrusion 346 can be generally aligned with the first side wall 324. The first protrusion 346 can be along a first side of the spinal implant device 300.

The body structure 302 can include a second protrusion 348. The second protrusion 348 can be near the second side wall 326. The second protrusion 348 can be coupled to the body structure 302. The second protrusion 348 can be integrally formed with the body structure 302. The second protrusion 348 and the body structure 302 can be monolithically formed. The second protrusion 348 can be semi-cylindrical. The second protrusion 348 can extend vertically. The second protrusion 348 can extend along the height of the body structure 302. The second protrusion 348 can be spaced inward from the proximal surface 342. The second protrusion 348 can be spaced inward from the second side wall 326. The second protrusion 348 can be generally aligned with the second side wall 326. The second protrusion 348 can be along a second side of the spinal implant device 300.

The first protrusion 346 and the second protrusion 348 can be the same or similar. The first protrusion 346 and the second protrusion 348 can have the same diameter. The first protrusion 346 and the second protrusion 348 can be disposed within the body structure 302. The first protrusion 346 and the second protrusion 348 can be diametrically opposed. The body structure 302 can be symmetrical. The body structure 302 can have right-left symmetry. The body structure 302 can have top-bottom symmetry. The body structure 302 can have two planes of symmetry.

The movable door 304 can include a distal surface 352. The distal surface 352 can be a flat surface or generally flat surface. The distal surface 352 can include the opening 316. The distal surface 352 can include the one or more features 318.

The movable door 304 can include a first tab 354. The first tab 354 can be near the first side wall 324. The first tab 354 can be near a first side of the spinal implant device 300. The first tab 354 can include a first passageway 356. The first passageway 356 can form a portion of an arc. The first passageway 356 can correspond to the outer surface of the first protrusion 346. The first passageway 356 can engage the first protrusion 346. The first passageway 356 can form a snap fit with the first protrusion 346. The first tab 354 can be coupled with the movable door 304. The first tab 354 can be integrally formed with the movable door 304. The tab first 354 and the movable door 304 can be monolithically formed. The first passageway 356 can be cylindrical. The first passageway 356 can extend vertically. The first passageway 356 can extend along the height of the movable door 304. The first tab 304 can be spaced outward from the distal surface 352. The first passageway 356 can be spaced outward from the distal surface 352. The first tab 354 can be generally aligned with the first side wall 324. The first tab 354 can be along a first side of the spinal implant device 300.

The first passageway 356 can include a first catch 362. The first catch 362 can limit or prevent further translational movement of the moveable door 304 relative to the first side wall 324. The first catch 362 can abut the first protrusion 346 to limit movement. The first catch 362 of the movable door 304 can contact the first protrusion 346 along the first side wall 324 when the movable door 304 is fully closed. The first protrusion 346 can interact with the first catch 362. The first catch 362 can prevent further movement of the movable door 346 when the first catch 362 is engaged. In some embodiments, the first catch 362 of the movable door 304 and the first protrusion 346 of the body structure 302 interlock together. The first catch 362 can provide tactile feedback that the movable door 304 is closed. The first catch 362 can form a snap fit.

The movable door 304 can include a second tab 358. The second tab 358 can be near the second side wall 326. The second tab 358 can be near a second side of the spinal implant device 300. The second tab 358 can include a second passageway 360. The second passageway 360 can form a portion of an arc. The second passageway 360 can correspond to the outer surface of the second protrusion 348. The second passageway 360 can engage the second protrusion 348. The second passageway 360 can form a snap fit with the second protrusion 348. The second tab 358 can be coupled with the movable door 304. The second tab 358 can be integrally formed with the movable door 304. The tab second 358 and the movable door 304 can be monolithically formed. The second passageway 360 can be cylindrical. The second passageway 360 can extend vertically. The second passageway 360 can extend along the height of the movable door 304. The second tab 358 can be spaced outward from the distal surface 352. The second passageway 360 can be spaced outward from the distal surface 352. The second tab 358 can be generally aligned with the second side wall 326. The second tab 358 can be along a second side of the spinal implant device 300.

The second passageway 360 can include a second catch 364. The second catch 364 can limit or prevent further translational movement of the moveable door 304 relative to the second side wall 326. The second catch 364 can abut the second protrusion 348 to limit movement. The second catch 364 of the movable door 304 can contact the second protrusion 348 along the second side wall 326 when the movable door 304 is fully closed. The second protrusion 348 can interact with the second catch 364. The second catch 364 can prevent further movement of the movable door 346 when the second catch 364 is engaged. In some embodiments, the second catch 364 of the movable door 304 and the second protrusion 348 of the body structure 302 interlock together. The second catch 364 can provide tactile feedback that the movable door 304 is closed. The second catch 364 can form a snap fit.

The passageway 356, 360 and the protrusion 346, 348 can have the same or similar diameter. The tabs 354, 358 can be formed with the movable door 304. The tabs 354, 358 can be diametrically opposed. The movable door 304 can be symmetrical. The movable door 304 can have right-left symmetry. The movable door 304 can have top-bottom symmetry. The spinal implant device 300 can have two planes of symmetry. The tabs 354, 358 can form a hinge with the protrusion 346, 348. The tabs 354, 358 can form a snap fit with the protrusion 346, 348.

The movable door 304 can be coupled near the first side wall 324 by engaging the first tab 354 and the first protrusion 346. The first protrusion 346 slides into the first passageway 356. The first protrusion 346 engages the first catch 362. The movable door 304 can be coupled near the second side wall 326 by engaging the second tab 358 and the second protrusion 348. The second protrusion 348 slides into the second passageway 360. The second protrusion 348 engages the second catch 364. The movable door 304 can be snapped onto the body structure 302.

In some embodiments, the movable door 304 can include a snap fit. The snap fit can attach the movable door 304 to the body structure 302. The movable door 304 can include the first tab 354 and the second tab 358. The tabs 354, 358 can be configured to flex to engage the protrusions 346, 348. The tabs 354, 358 can be configured to interlock with the protrusions 346, 348. In some embodiments, the tabs 354, 358 and protrusions 346, 348 can reversibly engage. The tabs 354, 358 and protrusions 346, 348 can snap together repeatedly. In some embodiments, the tabs 354, 358 can irreversibly or permanently engage. The tabs 354, 358 and protrusions 346, 348 can snap together once. The tabs 354, 358 and protrusions 346, 348 can snap together and be difficult to disengage. The tabs 354, 358 and protrusions 346, 348 can snap together and form a flush surface. The tabs 354, 358 and protrusions 346, 348 can snap together such that the tabs 354, 358 are flush with the side walls 324, 326. The tabs 354, 358 and protrusions 346, 348 can snap together such that the tabs 354, 358 are flush with the upper wall 330. The tabs 354, 358 and protrusions 346, 348 can snap together such that the tabs 354, 358 are flush with the lower wall 336. The tabs 354, 358 and protrusions 346, 348 can snap together such that movable door 304 is flush with the body portion 302.

In some embodiments, the movable door 304 does not rotate or pivot. In some embodiments, the movable door 304 snaps on by pushing the movable door 304 toward the distal end 310. The movable door 304 can be pushed along the longitudinal axis of the spinal implant device 300. The movable door 304 including one or more tabs 354, 358 can be formed in a number of ways. The body structure 302 including one or more protrusions 346, 348 can be formed in a number of ways. The movable door 304 and the body structure 302 can be 3D printed. The tabs 354, 358 can be integrally formed within the movable door 304. The protrusions 346, 348 can be integrally formed within the body structure 302.

In some embodiments, the spinal implant device 300 can include tabs 354, 358. The first tab 354 can be located along the first side wall 324. The second tab 358 can be located along the second side wall 326. The first passageway 356 can have a recessed shape. The first tab 354 can include a recessed portion that extends inward. The user can apply a force to the movable door 304. The tab 354 can facilitate the application of force to engage the first passageway 356 of the movable door 304 with the first protrusion 346. The tab 358 can facilitate the application of force to engage the second passageway 360 of the movable door 304 with the second protrusion 348. In some embodiments, the first tab 354 overhangs the first side wall 324. In some embodiments, the second tab 358 overhangs the second side wall 326. In some embodiments, the first tab 354 and the first protrusion 346 can disengage to open the movable door 304. The first tab 354 and the first protrusion 346 can engage to close the movable door 304. In some embodiments, the second tab 358 and the second protrusion 348 can disengage to open the movable door 304. The second tab 358 and the second protrusion 348 can engage to close the movable door 304.

The movable door 304 and the body structure 302 can have a complementary shape. In some embodiments, the distal surface 352 of the movable door 304 can abut the proximal surface 342 of the body structure 302 along the entire height of the movable door 304, or a portion thereof, when the movable door 304 is closed. In some embodiments, the distal surface 352 of the movable door 304 can abut the proximal surface 342 of the body structure 302 along the entire width of the movable door 304, or a portion thereof, when the movable door 304 is closed. The body structure 302 can form a support surface that mirrors the shape of the movable door 304. In some embodiments, the movable door 304 is spaced apart from the proximal surface 342 of the body structure 302 when the movable door 304 is closed. In some embodiments, the movable door 304 contacts the first side wall 324 when the movable door 304 is closed. In some embodiments, the first tab 354 contacts the first protrusion 346 when the movable door 304 is closed. In some embodiments, the movable door 304 contacts the second side wall 326 when the movable door 304 is closed. In some embodiments, the second tab 358 contacts the second protrusion 348 when the movable door 304 is closed.

The body structure 302 and the movable door 304 can snap together. The body structure 302 and the movable door 304 can form one or more flush surfaces. The body structure 302 and the movable door 304 can provide tactile feedback that the movable door 304 is closed. The movable door 304 and the body structure 302 can have mating surfaces. The movable door 304 and the body structure 302 can have interlocking features. The body structure 302 can form the recessed portion to accommodate the tabs 354, 358.

The movable door 304 and the body structure 302 together can form the upper surface of the spinal implant device 300. The upper surface of the spinal implant device 300 can include the upper wall 330 and the movable door 304. The movable door 304 and the body structure 302 together can form the lower surface of the spinal implant device 300. The lower surface of the spinal implant device 300 can include the lower wall 336 and the movable door 304. The movable door 304 and the body structure 302 together can form the first side surface of the spinal implant device 300. The first side surface of the spinal implant device 300 can include the first side wall 324 and the movable door 304. The first side surface of the spinal implant device 300 can include the first tab 354. The movable door 304 and the body structure 302 together can form the second side surface of the spinal implant device 300. The second side surface of the spinal implant device 300 can include the second side wall 326 and the movable door 304. The second side surface of the spinal implant device 300 can include the second tab 358.

In some embodiments, the upper wall 330 is flush with the movable door 304 when the movable door 304 is closed. In some embodiments, the lower wall 336 is flush with the movable door 304 when the movable door 304 is closed. In some embodiments, the first side wall 324 is flush with the movable door 304 when the movable door 304 is closed. In some embodiments, the second side wall 326 is flush with the movable door 304 when the movable door 304 is closed. The movable door 304 can extend across the entire width of the spinal implant device 300. The movable door 304 can extend across the entire height of the spinal implant device 300. The movable door 304 can extend across a portion of the length of the spinal implant device 300.

In some embodiments, the moveable door 304 can open by being pulled proximally relative to the body structure 302. In some embodiments, the moveable door 304 can close by being pushed distally relative to the body structure 302. The moveable door 304 can be aligned with the first side wall 324 when closed. The moveable door 304 can be aligned with the second side wall 326 when closed.

The movable door 304 can generally align with the taper of the upper wall 330. The movable door 304 can generally align with the taper of the lower wall 336. The movable door 304 can generally align with the taper of the side walls 324, 326. The plurality of ridges 334 can extend along a portion of the top surface of the spinal implant device 300. The plurality of ridges 334 can extend along a portion of the lower surface of the spinal implant device 300.

The spinal implant device 300 can include the cavity 306. In some embodiments, the movable door 304 can define the back inner surface of the cavity 306. The movable door 304 can close the opening 344 of the proximal surface 342 of the body structure 302. In some embodiments, the distal end 310 can define the front inner surface of the cavity 306. In some embodiments, the two opposing side walls 324, 326 can define the side inner surfaces of the cavity 306. In some embodiments, the upper wall 330 can define the upper inner surface of the cavity 306. In some embodiments, the lower wall 336 can define the lower inner surface of the cavity 306.

In some embodiments, the spinal implant device 100, 200, 300 are intervertebral body fusion devices for use in spine surgery. The spinal implant device 100, 200, 300 may also be referred to as interbody fusion devices or interbody cages. The spinal implant device 100, 200, 300 can be configured for placement between adjacent vertebrae. The spinal implant device 100, 200, 300 can be configured for placement between two cervical vertebrae. The spinal implant device 100, 200, 300 can be configured for placement between two lumbar vertebrae. The spinal implant device 100, 200, 300 can be configured for placement between two thoracic vertebrae. The spinal implant device 100, 200, 300 can be designed for any surgical approach. The spinal implant device 100, 200, 300 can be designed for an anterior approach. The spinal implant device 100, 200, 300 can be designed for a posterior approach. The spinal implant device 100, 200, 300 can be designed for minimally invasive surgery. The spinal implant device 100, 200, 300 can be designed for ALIF. The spinal implant device 100, 200, 300 can be designed for ACDF. The spinal implant device 100, 200, 300 can be generally wedge-shaped. The spinal implant device 100, 200, 300 can incorporate porous or network surfaces 128, 132, 138, 228, 232, 238, 328, 332, 338 on the upper, lower, and/or lateral sides. The porous or network surfaces 128, 132, 138, 228, 232, 238, 328, 332, 338 allow for bone to grow into and through the spinal implant device 100, 200, 300 and reduce the risk of subsidence into the vertebral bodies. The porous or network surfaces 128, 132, 138, 228, 232, 238, 328, 332, 338 can improve visualization radiographically so that fusion may be assessed. The upper and lower surfaces of the devices include ridges, teeth, or other generally sharp engagement members 134, 234, 334 to help prevent device migration in situ.

The spinal implant device 100, 200, 300 includes the movable door 104, 204, 304 to facilitate installation of bone graft material. The movable door 104, 204 can be pivoted through a range of motion. The movable door 304 can be removed. The movable door 104, 204 can have a range of motion of 15 degrees, 30 degrees, 45 degrees, 60 degrees, 75 degrees, 90 degrees, 105 degrees, 120 degrees, 135 degrees, 150 degrees, 165 degrees, 180 degrees, or any range of two of the foregoing values. The movable door 104, 204 can include a hinge pin 148, 248 that runs through a portion of the device.

In some embodiments, the spinal implant device 100, 200, 300 is 3D-printed (i.e., additively manufactured). In some embodiments, the spinal implant device 100, 200, 300 is manufactured as one piece and does not allow for disassembly. The movable door 104, 204 can be permanently coupled to the body structure 102, 202. The hinge pin 148, 248 can be disposed in a corresponding lumen 160, 260 through 3D-printing. The hinge pin 148, 248 can be captive. The spinal implant device 100, 200 can include tabs 154, 254 that close against the first pin 146, 246. The spinal implant device 300 can include tabs 354, 358 that close against the first protrusions 346, 348. The spinal implant device 100, 200, 300 can include passageways 156, 256, 356, 360 that can increase the manufacturing tolerance for the snap fit features so they may be printed as opposed to being precision machined after printing.

The spinal implant device 100, 200, 300 can be available in a multitude of sizes to suit the individual pathology and anatomic condition of the patient. The spinal implant device 100, 200, 300 have the same footprints, heights, lordosis angle, and outer profile geometry based on the anatomy. The spinal implant device 100, 200, 300 can share the same instrument interface features for device insertion and positioning. The spinal implant device 100, 200, 300 can have similar proximal features including the opening 116, 216, 316, the one or more features 118, 218, 318, and the one or more undercuts 120, 220, 230 to facilitate insertion.

The spinal implant device 100, 200, 300 can have a maximum length of 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 20 mm, 21 mm, 22 mm, 23 mm, 24 mm, 25 mm, 26 mm, 27 mm, 28 mm, 29 mm, 20 mm, 30 mm, 31 mm, 32 mm, 33 mm, 34 mm, 35 mm, between 6 mm and 10 mm, between 10 mm and 15 mm, between 15 mm and 20 mm, between 23 mm and 27 mm, between 25 mm and 29 mm, between 27 mm and 31 mm, between 29 mm and 33 mm, greater than 10 mm, greater than 15 mm, greater than 20 mm, greater than 23 mm, greater than 25 mm, greater than 27 mm, greater than 29 mm, greater than 31 mm, less than 31 mm, less than 29 mm, less than 27 mm, less than 25 mm, less than 20 mm, less than 15 mm, or any range of two of the foregoing values..

In some embodiments, the spinal implant device 100, 200, 300 can have a width of 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, 25 mm, 25.5 mm, 26 mm, 26.5 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, 35 mm, 35.5 mm, 36 mm, 36.5 mm, 37 mm, 37.5 mm, 38 mm, 38.5 mm, 39 mm, 39.5 mm, 40 mm, 40.5 mm, 41 mm, 41.5 mm, 42 mm, 42.5 mm, 43 mm, 43.5 mm, 44 mm, 44.5 mm, 45 mm, between 10 mm and 15 mm, between 14 mm and 16 mm, between 16 mm and 18 mm, between 17 mm and 20 mm, between 20 mm and 22 mm, between 28 mm and 32 mm, between 35 mm and 39 mm, between 39 mm and 43 mm, between 30 mm and 37 mm, between 30 mm and 41 mm, or any range of two of the foregoing values, greater than 10 mm, greater than 15 mm, greater than 20 mm, greater than 25 mm, greater than 30 mm, greater than 35 mm, greater than 40 mm, less than 43 mm, less than 41 mm, less than 39 mm, less than 37 mm, less than 35 mm, less than 30 mm, less than 22 mm, less than 20 mm, less than 15 mm, or any range of two of the foregoing values.

In some embodiments, the spinal implant device 100, 200, 300 can have a height of 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, between 5 mm and 12 mm, between 5 mm and 11 mm, between 6 mm and 11 mm, between 14 mm and 16 mm, between 16 mm and 18 mm, between 17 mm and 20 mm, between 10 mm and 14 mm, 10 mm and 15 mm, 10 mm and 20 mm, between 12 mm and 20 mm, 15 mm and 20 mm, greater than 3 mm, greater than 6 mm, greater than 10 mm, greater than 12 mm, greater than 14 mm, greater than 16 mm, greater than 18 mm, greater than 20 mm, less than 22 mm, less than 20 mm, less than 15 mm, less than 10 mm, or any range of two of the foregoing values. In some embodiments, the heights are anterior heights. In some embodiments, the heights are posterior heights.

In some embodiments, the spinal implant device 100, 200, 300 can have a lordosis angle of 0°, 1°, 2°, 3°, 4°, 5°, 6°, 7°, 8°, 9°, 10°, 11°, 12°, 13°, 14°, 15°, 16°,17°, 18°, 19°, 20°, between 0° and 5°, between 0° and 6°, between 0° and 7°, between 3° and 9°, between 5° and 7°, between 6° and 12°, between 8° and 16°, between 10° and 14°, between 12° and 14°, between 16° and 20°, approximately 6°, approximately 12°, approximately 18°, greater than 3°, greater than 6° greater than 7°, greater than 10°, greater than 15°, less than 20°, less than 16°, less than 12°, less than 10°, less than 8°, less than 6°, or any range of two of the foregoing values.

In some embodiments, the spinal implant device 100, 200, 300 can be positioned such that the movable door 104, 204, 304 and/or the upper wall 130, 230, 330 contact a vertebral end plate of a superior vertebra. In some embodiments, the spinal implant device 100, 200, 300 can be positioned such that the movable door 104, 204, 304 and/or the lower wall 136, 236, 336 contact a vertebral end plate of an inferior vertebra. In some embodiments, the spinal implant device 100, 200, 300 can be inserted in the opposite orientation. The spinal implant device 100, 200, 300 can include a retention feature that retains the moveable door 104, 204, 304 in a closed position.

In some embodiments, the spinal implant device 100, 200, 300 can have a slight inclination, called a lordosis angle. In some embodiments, the lordosis angle is approximately 7°. Other configurations are contemplated, for example 0°, 1°, 2°, 3°, 4°, 5°, 6°, 7°, 8°, 9°, 10°, 11°, 12°, 13°, 14°, 15°, 16°, 17°, 18°, 19°, 20°, between 4° and 6°, between 0° and 5°, between 3° and 5°, or any range of the foregoing values. In some embodiments, the spinal implant device 1100, 200, 300 can have a slight inclination, called a kyphosis angle. In some embodiments, the kyphosis angle is approximately 5°. Other configurations are contemplated, for example 0°, 1°, 2°, 3°, 4°, 5°, 6°, 7°, 8°, 9°, 10°, 11°, 12°, 13°, 14°, 15°, 16°, 17°, 18°, 19°, 20°, between 4° and 6°, between 0° and 5°, between 3° and 5°, or any range of the foregoing values.

The spinal implant device 100, 200, 300, or any portion thereof, can comprise any material described herein. The spinal implant device 100, 200, 300 can include or omit any feature or features described herein. A spinal implant device can comprise one or more features of any spinal implant device described herein.

The spinal implant device 100, 200, 300 can be manufactured through 3D printing. The spinal implant device 100, 200, 300 can be manufactured using additive layer manufacturing. In some embodiments, the spinal implant device 100, 200, 300 is constructed from a digital model using manufacturing processes where materials are added to create the three-dimensional shape of the spinal implant device. In some embodiments, the spinal implant device 100, 200, 300 is constructed layer by layer, enabling the one-piece design. In some embodiments, the spinal implant device 100, 200, 300 can include the movable door 104, 204, 304 manufactured by 3D printing. In some embodiments, the spinal implant device 100, 200 can include a movable joint manufactured by 3D printing. In some embodiments, the spinal implant device 100, 200 can include the pin 148, 248 and the lumen 160, 260 manufactured by 3D printing. In some embodiments, the spinal implant device 100, 200 can include a snap fit manufactured by 3D printing. In some embodiments, the spinal implant device 100, 200 can include the pin 146, 246 and the passageway 156, 246 manufactured by 3D printing. In some embodiments, the spinal implant device 100, 200 can include the catch 162, 262 manufactured by 3D printing. In some embodiments, the spinal implant device 300 can include the protrusion 346, 348 and the passageway 356, 360 manufactured by 3D printing. In some embodiments, the spinal implant device 300 can include the catch 362, 364 manufactured by 3D printing.

The method of manufacturing the spinal implant device 100, 200, 300 can include 3D printing a spinal implant device comprising a distal end, a proximal end, two opposing side walls extending between the distal end and the proximal end, an upper wall, and a lower wall forming a lower surface of the body structure 102, 202, 302. The method of manufacturing the spinal implant device 100, 200, 300 can include 3D printing a spinal implant device comprising the cavity 106, 206, 306. The method of manufacturing the spinal implant device 100, 200 can include 3D printing a spinal implant device comprising the movable door 104, 204 coupled to the proximal end of the spinal implant device by a movable joint. The method of manufacturing the spinal implant device 300 can include 3D printing a spinal implant device comprising the movable door 304 configured to be coupled to the proximal end of the spinal implant device by a snap fit. The method of manufacturing the spinal implant device 100, 200, 300 can include 3D printing a spinal implant device comprising the porous or network surface 128, 132, 138, 228, 232, 238, 328, 332, 338. The method of manufacturing the spinal implant device 100, 200 can include 3D printing, wherein the spinal implant device is 3D printed as one piece. The method of manufacturing the spinal implant device 300 can include 3D printing, wherein the spinal implant device is 3D printed as two interlocking pieces. The method of manufacturing the spinal implant device 100, 200 can include 3D printing a spinal implant device, wherein the movable joint comprises the pivot pin 146, 246 disposed within the lumen 160, 260. The method of manufacturing the spinal implant device 100, 200 can include 3D printing a spinal implant device wherein the movable door 104, 204 is configured to pivot to access the central cavity 106, 206.

In some embodiments, the spinal implant device 100, 200 comprises a hinged cage that is 3D printed as one piece. In some embodiments, the spinal implant device 300 comprises a snap fit cage that is 3D printed as two pieces. In some embodiments, the manufacturing method enables the designs described herein. In some embodiments, the spinal implant device 100, 200 would not be able to integrate the movable door 104, 204 with conventional manufacturing methods. In some embodiments, the spinal implant device 300 would not be able to snap fit the body structure 302 with the moveable door 304 without additional steps under conventional manufacturing methods.

The implant inserter can couple with the spinal implant device 100, 200, 300. In some methods of use, the spinal implant device 100, 200, 300 can be placed between the endplates of the superior and the inferior vertebra. In some embodiments, the spinal implant device 100, 200, 300 can be retained between the superior vertebra and the inferior vertebra.

The spinal implant device 100, 200, 300 can comprise any structure configured to maintain a separation and resist compression between two adjacent vertebral bodies. The spinal implant device 100, 200, 300 can have any of a variety of overall shapes, including but not limited to a rectangular box, a trapezoidal box, H-shaped, O-shaped, V-shaped, etc. The spinal implant device 100, 200, 300 can have an anterior or proximal surface, a superior or upper surface, an inferior or lower surface, side surfaces, and a posterior or distal surface. Each surface need not be flat and can be curved or any combination thereof. The upper and lower surfaces can be configured for facing the superior and inferior vertebral bodies. The side surfaces can taper inward from the proximal end to the distal end. The distal end of each side surface can be tapered to facilitate insertion.

The proximal end 114, 224, 334 can have a generally flat configuration, curved configuration, or combination thereof. The proximal end 114, 224, 334 can form the undercut 120, 220, 320. The edges of the proximal end 114, 224, 334 can optionally be angled, rounded, or curved. The edges of the proximal end 114, 224, 334 can be smoothed or polished. In some embodiments, the proximal end 114, 224, 334 can have a generally square or rectangular shape. In other embodiments, the proximal end 114, 224, 334 can comprise any of a variety of other shapes, including trapezoids, circles, ovals, polygons, or other closed shapes. The proximal end 114, 224, 334 can be dimensioned to allow stable connection to the inserter as described herein.

The spinal implant device 100, 200, 300 can have any number of lumens or pores e.g., one, two, three, four, five, etc. The porous or network surfaces 128, 132, 138, 228, 232, 238, 328, 332, 338 can include lumens. The lumens can extend between any surface of the spinal implant device 100, 200, 300. The lumens may be oriented in different directions. The lumens may be oriented between the upper wall 130, 230, 330 and the lower wall 136, 236, 336. The lumens may be oriented between the side walls 124, 226, 224, 226, 324, 326. The lumens may be oriented between the distal end 110, 210, 310 and the proximal end 114, 214, 314. The lumens may promote bone ingrowth between the superior vertebra and the inferior vertebra. The lumens may allow visualization of bony ingrowth. The lumens can allow bony growth into the cavity 106, 206, 306 of spinal implant device 100, 200, 300. The lumens can be filled with graft materials. The graft material can be an autograft, allograft, xenograft, or synthetic material. Synthetic graft material can be ceramic-based, silicon-based, or calcium-based. The graft material can also include osteoinductive factors to promote bone ingrowth. One skilled in the art will appreciate that there are many varieties of synthetic graft materials and constituents that can be used.

Each lumen need not have the same configuration or size. In some embodiments, the lumens can be round in cross-sectional shape. In some embodiments, the lumens can comprise any of a variety of shapes including squares, rectangles, trapezoids, circles, ovals, polygons, or other closed shapes. In some embodiments, at least a portion of the lumens can have a non-round cross-sectional shape.

The relative configuration of the upper surface and the lower surface can vary, depending upon the relative position desired between the two adjacent vertebrae, the anatomical shape of the vertebrae, ease of insertion of the spinal implant device 100, 200, 300, and other factors. For example, if a neutral vertical alignment is desired between two vertebrae, the upper and lower surface can have generally parallel planar orientations. If a non-neutral alignment is desired, for instance, to maintain a natural spinal curvature in the region of the spine, the upper and lower surface can have a wedge-like relationship to allow fixation of the vertebrae in the desired non-neutral position. A non-neutral alignment with respect to the anterior-posterior direction can also be used to compensate for excessive lordosis or kyphosis in other portions of the vertebral column. The height of the spinal implant device 100, 200, 300 at any section between the upper and lower surfaces can be further configured to accommodate degenerative changes or anatomical anomalies to provide fixation in the desired relative position. Likewise, the side walls of the spinal implant device 100, 200, 300 can be generally skewed. In some embodiments, the side walls of the spinal implant device 100, 200, 300 taper with increasing distance from the posterior end 114 of the spinal implant device 100, 200, 300. A tapered spinal implant device 100, 200, 300 can facilitate insertion of the spinal implant device 100, 200, 300 into the intervertebral space. In some embodiments, the one or more side walls can flare distally or have both tapering and flaring portions.

One or more surfaces of the spinal implant device 100, 200, 300 can have surface projections, indentations, or holes or pores that can further alter the characteristics of the spinal implant device 100, 200, 300. The spinal implant device 100, 200, 300 can include one or more ridges 134, 234, 334 designed to engage the adjacent anatomical features. The ridges 134, 234, 334 can include angled projections, barbs, teeth, or ramped surfaces which incline outwardly from one or more surface of the spinal implant device 100, 200, 300. In some embodiments, the ridges 134, 234, 334 are provided on the upper wall 130, 230, 330, the lower wall 136, 236, 336, and/or the movable door 104, 204, 304. Other surfaces of the spinal implant device 100, 200, 300 can also include one or more ridges 134, 234, 334. In some embodiments, the ridges 134, 234, 334 can be combined with indentations or holes or pores for allowing bony ingrowth which may also enhance insertion and stabilization of the spinal implant device 100, 200, 300.

The ridges 134, 234, 334 can allow insertion of the spinal implant device 100, 200, 300 in one direction but resist movement in the opposite direction. The ridges 134, 234, 334 can be advantageous in reducing the migration of the spinal implant device 100, 200, 300 out of the intervertebral space. The ridges 134, 234, 334 can maintain the position of the spinal implant device 100, 200, 300.

The spinal implant device 100, 200, 300 can include, be made of, treated, coated, filled, used in combination with, or contain artificial or naturally occurring materials suitable for implantation in the human spine. These materials can include any source of osteogenic, bone growth-promoting materials, bone derived substances, bone morphogenetic proteins, hydroxyapatite, genes coding for the production of bone, and bone, including but not limited to cancellous bone and/or cortical bone. The spinal implant device 100, 200, 300 can be formed of various materials, including but not limited to titanium and its alloys surgical grade plastics, plastic composites, ceramics, or other materials suitable for use as a spinal fusion implant. In some embodiments, the spinal implant device 100, 200, 300 can comprise a radiolucent material, a radio-opaque material, or a combination thereof. A spinal implant device 100, 200, 300 that is partially or completely radiolucent can be advantageous when evaluating the effect of the spinal implant device 100, 200, 300 post-implantation. Many existing interbody implants obscure visualization of the vertebrae, which can complicate postoperative treatment, diagnosis, and prognosis of the patient’s condition. In some embodiments, the spinal implant device 100, 200, 300 can include materials that are bioabsorbable in the body.

The spinal implant device 100, 200, 300 of the described embodiments can be formed of a porous material or can be formed of a material that intrinsically participates in the growth of bone from one of adjacent vertebral bodies to the other of adjacent vertebral bodies. The spinal implant device 100, 200, 300 can be treated with, coated with, or used in combination with substances to inhibit scar tissue formation. The spinal implant device 100, 200, 300 can be modified, or used in combination with materials to provide antibacterial properties, such as, but not limited to, electroplating or plasma spraying with silver ions or other substance. The spinal implant device 100, 200, 300 can optionally comprise an electrical source to provide iontophoresis of silver ions into the surrounding tissue to prevent infection. The antibacterial properties can include bactericidal and/or bacteriostatic characteristics. Similarly, anti-fungal characteristics can also be provided.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described, which will be within the full intended scope of the invention as defined by the following appended claims. Although the present invention has been described in relation to various exemplary embodiments, various additional embodiments and alterations to the described embodiments are contemplated within the scope of the invention. Thus, no part of the foregoing description should be interpreted to limit the scope of the invention as set forth in the following claims. For all of the embodiments described above, the steps of the methods need not be performed sequentially.

What is claimed is:

1. A spinal implant device comprising:
a body structure,
a movable door coupled or configured to couple to the body structure,
wherein the spinal implant device comprises a distal end, a proximal end, two opposing side walls extending between the distal end and the proximal end, an upper wall, a lower wall, and a central cavity,
wherein the movable door is configured to open to allow the central cavity to be packed with material, wherein the movable door is configured to close.

2. The spinal implant device of claim 1, wherein the two opposing side walls comprise porous or network surfaces.

3. The spinal implant device of claim 1, wherein the upper wall and the lower wall comprise porous or network surfaces.

4. The spinal implant device of claim 1, wherein the proximal end comprises a threaded opening.

5. The spinal implant device of claim 1, wherein the upper wall and the lower wall comprise ridges.

6. The spinal implant device of claim 1, wherein the body structure comprises a first pin and a second pin.

7. The spinal implant device of claim 1, wherein the movable door is coupled to the body structure with a hinge.

8. The spinal implant device of claim 1, wherein the movable door is coupled to the body structure with a pin extending vertically between the upper wall and the lower wall.

9. The spinal implant device of claim 1, wherein the movable door is configured to pivot toward and away from the body structure.

10. The spinal implant device of claim 1, wherein the movable door is coupled to the body structure with a pin extending horizontally between the movable door and the body structure.

11. The spinal implant device of claim 1, wherein the movable door is configured to pivot from a generally transverse orientation to a generally vertical orientation.

12. The spinal implant device of claim 1, wherein the body structure comprises a captive pin formed by 3D printing.

13. The spinal implant device of claim 1, wherein the movable door comprises a catch configured to engage a pin when the moveable door is closed.

14. The spinal implant device of claim 1, wherein the movable door comprises a hinge aligned along a side wall of the two opposing side walls.

15. The spinal implant device of claim 1, wherein the body structure comprises a first protrusion and a second protrusion for securing the movable door.

16. The spinal implant device of claim 15, wherein the movable door comprises a first tab and a second tab configured to engage the first protrusion and the second protrusion when the moveable door is closed.

17. The spinal implant device of claim 15, wherein the movable door comprises a first catch and a second catch configured to form a snap fit with the first protrusion and the second protrusion when the moveable door is closed.

18. A method comprising:
providing a spinal implant device of claim 1;
packing the cavity with the material;
closing the movable door;
inserting the spinal implant device between adjacent vertebrae.

19. The method of claim 18, wherein the material is a graft material.

20. A method of manufacturing a spinal implant device comprising:
3D printing a spinal implant device comprising:
a body structure,
a movable door coupled to the body structure,
wherein the spinal implant device comprises a distal end, a proximal end, two opposing side walls extending between the distal end and the proximal end, an upper wall, a lower wall, and a central cavity,
wherein the movable door is configured to open to allow the central cavity to be packed with material, wherein the movable door is configured to close,
wherein the spinal implant device is 3D printed as one piece.
